SEQUENCE LISTING (49 sequences)

SEQ ID NO: 1 - protein, 228 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFG

SEQ ID NO: 2 - protein, 322 aa, Photinus pyralis
NQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 3 - DNA, 966 bp, Photinus pyralis
aatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattg

SEQ ID NO: 4 - protein, 228 aa, Photinus pyralis
MEDAKNIKKGPAPFYPLEDGTAGEQLHKAMKRAALVPGTIAFTDAAIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFG

SEQ ID NO: 5 - DNA, 684 bp, Photinus pyralis
atggaagacgccaaaaacataaagaaaggcccggcgccattctatcctctagaggatggaaccgctggagagcaactgcataaggctatgaagagagcggccctggttcctggaacaattgcttttacagatgcagcgatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggc

SEQ ID NO: 6 - protein, 12 aa, Photinus pyralis
DPIFGNQIIPDT

SEQ ID NO: 7 - protein, 228 aa, Photinus pyralis
MEDAKNIKKGPAPFYPLEDGTAGEQLHKAMKRYALVPGTIAFTDAAIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFG

SEQ ID NO: 8 - protein, 228 aa, Photinus pyralis
MEDAKNIKKGPAPFYPLEDGTAGEQLHKAMKRAALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFG

SEQ ID NO: 9 - protein, 8 aa, Photinus pyralis
RYALVPGT

SEQ ID NO: 10 - protein, 5 aa, Photinus pyralis
AHIEV

SEQ ID NO: 11 - protein, 48 aa, Photinus pyralis
SGSTGLPKGVALPHRTACVRFSHARDPIFGNQIIPDTAILSVVPFHHG

SEQ ID NO: 12 - protein, 47 aa, Unknown
  Rhagophthalmus ohbai
SSGTTGLPKGVVLTHRNLSVRFHCKDPLFGTRTIPSTSILSIVPFHH

SEQ ID NO: 13 - protein, 44 aa, Unknown
  Campontus floridanus
SGTTGLPKGVMWTDKNITTIIRMYINHPFVANAMSLALLPFFHA

SEQ ID NO: 14 - protein, 42 aa, Unknown
  Papilio machaon
SGTTGLPKGVMQTHLNALIVFNLSCLLDPTQIVLNVTSWYHA

SEQ ID NO: 15 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 16 - protein, 535 aa, Bombyx mori
MEKEKQNAGIIYGEQNFSFPNHMNYGKFLLERLSKEKAKVALINGASGEKLTFGEMTQQIVNIASAIKQLGIGKGDIVAICSENRTEYLTTTIAVLCTGATVTFINSAYSKNEFIHTTKISKPKYIFLSPSAYKEFYVTLKKLNIVQKFFLYGISDDKDVISFRDLATKYTHFNDFRPKDFNGTDSTSIILYSSGTTGLPKGVKLTHRNLLVSGLQKPFCQKDLVILTLAPWCNTVGIILTLSCLSKNRTVLYLNKFQEDVYLQCIEKYKAGILLMVPPLLVILTKSKLIDKYDVSSVQIMYCGGAPLDSSVMTSIKKRFTGLKHVLQGYGMTETTGALTEEREHSNKLGSVGKVVEGNIVKVVDVETRKTLGPNQNGEICVKGPVLFEDYIGKDINEDLDEDGFYKTGDIAYYDNEGYFYIVDRIKELIKYKAGQVAPSELEAILLQHDAVQDVGVAGAPDPLVGELPTAFVVKKPNSKVTEKELIDFVAARVSSWKQLRGGVRFVNEIPKTGSGKILRRILRDSLTKPPASKL

SEQ ID NO: 17 - protein, 536 aa, Unknown
  Papilio machaon
MLRNSKYLFSPDEHFVPSKLHLGDFFIKTFKKYGDKVALINGATDTAVTYNDLLQASMNISVSLSQLGLKKGETVGICSESRLETWSVIVGAVCAGGVMTPLNIRYVKDELRHVMNISKPKFLFCSKQAYDNHEAIFKSLVYVENIILFGDERVKGTLLYKDLAMISPNSMLKENVNFEEFEVVDVQGQIDTVFLMYSSGTTGLPKGVMQTHLNALIVFNLSCLLDPTQIVLNVTSWYHAMGFMSTFIYLSHGCKHIFLPKFETDLYLKTIEKYKVNLLVLVPPVLMTLIKTSSQYDLSSVVAIVSAAAALRKETIAAVNEKFKNLKGICQGYGMTETTFVVTMDTLFTDGTCKPGSVGKVVPNTLIKIADVETREPLGPYQNGEVCIKSPLILKDYIGHDKNCCLDKDGFFRTGDVGYYDEDKHLFIVDRIKELIKYKAYQVPPAEIEAVLLQHEGVRDVGVVGLPHPEAGELPVAFVVPQAGANPTEAELQHFVAERLSNPKHLRGGVHFVKEIPKTASGKILRKELRELAINF

SEQ ID NO: 18 - protein, 531 aa, Unknown
  Danaus plexippus plexippus
MLRNSNYVYGDRSLSVPADLNFGSYIIDAIWKHGDKTAMTNCATNQCLSYRAMAVEAMNLSISLTARGVKRGDVIAISSENRMEYFPTLLGICCTGATVTTTNPTYTKDEHKYVLDISKPKLVFCSPSAYKAHGKLFHSLPYIKSVVVYGERIPNTLSYEDLIRDNGVITPENFMAKDVEGQNDAAIVLYSSGTTGLPKGVMLTHLNVIVTCLVAPSVDPNELTLYITPWYHSMGLIASLRVFSKGNVMLFLPKFECDQYLRTIENYKVGQLVVAPPVLVALSKYSAKYDVSSVVAAFSGAAPLKKETIDAAKKRFPNLKHVFQGYGMTETTFSVIRDTYDSAHLSKTGGAGTIAACVVIKIVDIETRRPLGPNCRGEICVKGAPIMRGYVGRDRGDDFDDEGFFKTGDIGYYDDDKYFFVVDRLKELIKYKGYQVPPAEIEAVLTQHPDILEAGVVGVPHEGGEAPLAFVARRPGSNLTVEEVKSYVAEKLSNPKRLRGGVRFVEDIPKNPSGKILRKKLRGMLKNKSKL

SEQ ID NO: 19 - protein, 542 aa, Unknown
  Photophorus jansonii
MKREKNVVYGPEPLQPLENITAGEMLYKALKRHSHLPQALVDVIGQESLSYKEFFDLTCQLGQSLHNCGYKLSDVVSICAENNKRFFVPIIAAWYVGMIVAPVNEDYIPDELCKVMGISKPILVFTTRKILPKVLEVQKRLSHIKRIIILDSPENLHGCECLSNFIARFSDGHTTFFKPMHYDPAEQVAAILCSSGTTGLPKGVMQTHRNICVRLLHASDPRSGTQLIPGVSVLAYLPFFHAFGFSINLGYFMVGLRIIMLRRFDQETFLKAIQDYEVRSVINVPAIILFLSKSPLVDKYDLSSLKELCCGAAPLAREIGELAAKRLNLPGVRCGFGLTESTSANIHSLPDYIKPGSLGTVTPLMKVKILNRDTGEALGPNQVGELCVKGPMVSKGYVNNIEATKEAIDEDGWLHSGDFGYYDEEEHFYVVDRYKELIKYKGYQVAPAELEEILLKHPCIRDAAVVGIPDVAAGELPSAFVVKQPGKEVTEKEVYDFLAERVSHSKYLRGGVRFVDEIPRNVTGKITRKELLKQLLNKTSKL

SEQ ID NO: 20 - protein, 543 aa, Pyrophorus plagiophthalamus
MMKREKNVIYGPEPLHPLEDKTAGEMLFRALRKHSHLPQAIVDVFGDESLSYKEFFEATCLLAQSLHNCGYKMNDVVSICAENNKRFFIPIIAAWYIGMIVAPVNESYIPDELCNVMGISKPQIVFCTKNILNKVLEVQSRTNFIKRIIILDTVENIHGCESLPNFISRYSDGNIANFKPLHYDPVEQVAAILCSSGTTGLPKGVMLTHQNICVRLIHALDPEAGTQLIPGVTVLVYVPFFHAFGFGINLGYFMVGLRVIMLRRFEQEAFLKAIQDYEVRSIVNVPAIILFLSKSPLVDKYDLSSLRELCCGAAPLAKEVAEIAVKRLNLPGIRCGFGLTESTSANIHSLGDEFKSGSLGRVTPLMAAKIADRETGKALGPNQVGELCIKGPMVSKGYVNNVKATKEAIDDDGWLHSGDFGYYDEDEHFYVVDRYKELIKYKGSQVAPAELEEILLKNPCIRDVAVVGIPDLEAGELPSAFVVIQPGKEITAKEVYDYLAERVSHTKYLRGGVRFVDSIPRNVTGKITRKELLKQLLEKSSKL

SEQ ID NO: 21 - protein, 541 aa, Unknown
  Pyrophorus angustus luscus
MDKEKYILHGPESIWYVPKTTAGQQVYNALVRHSHLPEAMIDAHIQQKVSYKELLETTCRLAQSLQRCGYKQNDVISICSENNLNFHCPAIAALYLGIITAPLEGYIEGELHNALNLSKPKLIFCSTKLLPKMQAMKQKFAFIKKLIILDVDEDIGSNESLSNFILRNSDASYKNFRPLDFDSNEQVAFILCSSGTTGLPKGVMLTHTNIAVRFAHARDPRIGTQTIPGTTVLSFMPFFHALGFITTLEYFLLGLRVIMLKKFDSELFLKSIQDYEVRSMIIVPLIVSFLAKSPLVDKYDLSSLKQISCGAAPLGKEVGDALLKRLNLEGISQGYGLTELTVAVTLTPDNEFRPGSSGAVVPFMSAKVIDNDTGKPLGPGVTGELYFKGGLVMKGYVGNISATKEMIDENGWLRTGDLGYYDKEGHFYIKGRLKELIKYKGFPVPPAELEALLLTHPCIKEAAVIGIPDKSAGELPAAFVVKQPGKQITEKEVYDFVAGQISSPKHLRGGVRFIDEIPKNATNKIKRDVLRDLVTKMKSKL

SEQ ID NO: 22 - protein, 544 aa, Unknown
  Agrypnus binodulus binodulus
MSKESNIVYGPVGAAPVLESTAGKQLFDSLKRHGHLPQAIIDYQTKQSISYKNLFEATCKLAHSLEEYGLKQNDVIAICSENNLNFYKPVCAALYCGIVIAPLNDSYSEGEYVNALNISEPKLIFCSKKCLPRLVGLKARCSFIKGFVVIDSTEDINGNECLPNFILRNSDPNFDIEKYEPRVFNSNEQVAAILLSSGTTGFPKGVMLTHKNFSILFAHANDPVSGTQRIPGTTVLSILPYFHGFGFITNISYIKSGIRVVMLQRFEPEAFLRAIEEYEVRSTITVPPILIFLAKSPIVDKYNLSSLKEIICGAAPSGREIVEAVVKRLKVSGIRYGYGLTECGLAICTTPPNNFKIGSSGVVVPFMAVKIRDVESGKTLKPTQIGEICVKGDMLMKGYAGNEKATKEMIDEDGWLHTGDIGYFDKDGHIYIVDRIKELIKYKGFQVPPAELEALLLHHPCVKDAAVIGIPDELAGELPAAFIVKQHGKEVTEKEIVDYIAKQVSSAKHLRGGVRFIPDIPRTAAGKIQRNLLRNMIAKKKSKL

SEQ ID NO: 23 - protein, 543 aa, Unknown
  Rhagophthalmus ohbai
MPNEIILHGAKPRDPLDLGTAGIQLYRALTNFSFLREALIDAHTEEVVSYADILENSCRLAKCYENYGLRQNSVISVCSENSTIFFYPVIAALYMGVITATVNDSYTERELLETLNISKPELVFCSKKAIKNMMALKRNVNFIKKVVLLDSKEDMGEAQCLSNFMARYSEPNLDVRNFKPRDFDAKEQVALIMSSSGTTGLPKGVVLTHRNLSVRFVHCKDPLFGTRTIPSTSILSIVPFHHAFGMFTTLSYFIVGLRVVLLKRFEEKFFLSTIEKYRIPTIVLAPPVMVFLAKSPLVDQYDLSSIREVATGGAPVGTEVAVAVAKRLKIGGILQGYGLTETCCAVLITPHDDVKTGSTGRVAPYVQAKIVDLTTGKSLGPNKRGELCFKSEIIMKGYFNNKQATEEAIDKEGWLHSGDVGYYDDDGHFFVVDRLKELIKYKGYQVAPAELEWLLLQHPSIKDAGVTGVPDEAAGELPGACIVLQEGKSLTEQEIIDYIAERVSPTKRIRGGVVFVDDIPKGATGKLVRSELRKLLAQKKSKL

SEQ ID NO: 24 - protein, 545 aa, Unknown
  Phrixothrix vivianii
MEEENIRHGERPRDIVHPGSAGQQLYQSLYKFASFPEAIIDAHTNEVISYAQIFETSCRLAVSIEQYGLNENNVVGVCSENNINFFNPVLAALYLGIPVATSNDMYTDGELTGHLNISKPTIMFSSKKALPLILRVQQNLSFIKKVVVIDSMYDINGVECVSTFVARYTDHTFDPLSFTPKDFDPLEKIALIMSSSGTTGLPKGVVLSHRSLTIRFVHSRDPIYGTRTVPQTSILSLVPFHHAFGMFTTLSYFVVGLKVVMLKKFEGALFLKTIQNYKIPTIVVAPPVMVFLAKSPLVDQYDLSSLTEVATGGAPLGKDVAEAVAKRLKLPGIIQGYGLTETCCAVMITPHNAVKTGSTGRPLPYIKAKVLDNATGKALGPGERGEICFQSEMIMKGYYNNPEATIDTIDKDGWLHSGDIGYYDEDGNFFIVDRLKELIKYKGYQVAPAELENLLLQHPSIADAGVTGVPDEFGGQLPAACVVLESGKTLTEKEVQDFIAAQVTPTKHLRGGVVFVDSIPKGPTGKLIRKELREIFAQRAPKSKL

SEQ ID NO: 25 - protein, 545 aa, Unknown
  Brasilocerus sp.
MEEENIRYGDRPRDIVHPGSAGEQLYQSLYKFSYFPEAIVDAHTNEKISYATIFETSCRLAVSIEKLGLNERNVVGVCSESNLNFFNPVLAALYLGIPVATSNDMYTDGELSGHLNISKPDIMFCSKKALPLILRAQKNLNFIKKVVVIDSMFNIDGVECVHSFVSHYTDSSFDPLSFKPKQFDPLQEIALIMSSSGTTGLPKGVVLSHRSLTIRFVHSRDPLYGTRTIPETSILSLVPFHHAFGMFTTLSYFVVGLRIVMLKKFEGDLFLKTIQNYKIPTIVVAPPVMVFLAKSPLVDKYDLSSLREVATGGAPLGKDVGEAVAKRLGLSGVLQGYGLTETCCAVVITPHNNVKTGSAGKVVPYVSAKVLDKATGKALGPKERGEICFKSEMLMKGYYNNPEATRETIDEDGWLHSGDIGYYEEDGMFYIVDRLKELIKYKGYQVAPAELENLLLQHPDIADAGVTGIPDEFAGQLPAACVVLEPGKTLTEKEAMDFIAEQVTPTKHLRGGVIFVDRIPKGPTGKLIRNELRAIFAKKAASSKL

SEQ ID NO: 26 - protein, 547 aa, Unknown
  Taximastinocerus sp.
MEEENIMHGAEPRDLVHPGSAGTQLYRSLYKFSYFPEAIVDAHTHEVISYATIFNVSCRLAASLEKYGLGKEDDVVGVCSENNLNFFSPVLAALYLGVPVATSNDMYTDGEISGHLNISKPCIMFCSKKALPLILKVQKNLHFMKKVVVIDSMYDMNGVECINTFMNHYTDHTFDPLKFQPKEFDPLKKTALIMSSSGTTGLPKGVVLSHRSLTIRFVHSRDPIYGTRTIPETSILSLVPFHHAFGMFTTLSYFVVGLRIVMLKKFEGELFLKTIQHYKIPTIVVAPPVMVFLAKSPLVDKYDLSSLREVATGGAPIGKDVAHAVAKRLGLSGILQGYGLTETCCAVVITPHDNLRTGSAGKVVPYVKAKILDKATGKALGPNERGEICFKSEMLMKGYHNNPEATRETIDEDGWLHSGDIGYYEEDGTIYIVDRLKELIKYKGYQVAPAELENLLLQHPDIADAGVTGVPDGFAGQLPAACIVLEPGKTLTEKEVVDFLAERVTPTKYLRGGVIFVDRIPKGPTGKLMRQELRAIFAAKKAQTSKL

SEQ ID NO: 27 - protein, 548 aa, Unknown
  Aquatica lateralis
MENMENDENIVYGPEPFYPIEEGSAGAQLRKYMDRYAKLGAIAFTNALTGVDYTYAEYLEKSCCLGEALKNYGLVVDGRIALCSENCEEFFIPVLAGLFIGVGVAPTNEIYTLRELVHSLGISKPTIVFSSKKGLDKVITVQKTVTAIKTIVILDSKVDYRGYQSMDNFIKKNTPPGFKGSSFKTVEVNRKEQVALIMNSSGSTGLPKGVQLTHENAVTRFSHARDPIYGNQVSPGTAILTVVPFHHGFGMFTTLGYLTCGFRIVMLTKFDEETFLKTLQDYKCSSVILVPTLFAILNRSELLDKYDLSNLVEIASGGAPLSKEIGEAVARRFNLPGVRQGYGLTETTSAIIITPEGDDKPGASGKVVPLFKAKVIDLDTKKTLGPNRRGEVCVKGPMLMKGYVDNPEATREIIDEEGWLHTGDIGYYDEEKHFFIVDRLKSLIKYKGYQVPPAELESVLLQHPNIFDAGVAGVPDPIAGELPGAVVVLEKGKSMTEKEVMDYVASQVSNAKRLRGGVRFVDEVPKGLTGKIDGKAIREILKKPVAKM

SEQ ID NO: 28 - protein, 548 aa, Luciola cruciata
MENMENDENIVVGPKPFYPIEEGSAGTQLRKYMERYAKLGAIAFTNAITGVDYSYAEYLEKSCCLGKALQNYGLVVDGRIALCSENCEEFFIPVIAGLFIGVGVAPTNEIYTLRELVHSLGISKPTIVFSSKKGLDKVITVQKTVTTIKTIVILDSKVDYRGYQCLDTFIKRNTPPGFQASSFKTVEVDRKEQVALIMNSSGSTGLPKGVQLTHENTVTRFSHARDPIYGNQVSPGTAVLTVVPFHHGFGMFTTLGYLICGFRVVMLTKFDEETFLKTLQDYKCTSVILVPTLFAILNKSELLNKYDLSNLVEIASGGAPLSKEVGEAVARRFNLPGVRQGYGLTETTSAIIITPEGDDKPGASGKVVPLFKAKVIDLDTKKSLGPNRRGEVCVKGPMLMKGYVNNPEATKELIDEEGWLHTGDIGYYDEEKHFFIVDRLKSLIKYKGYQVPPAELESVLLQHPSIFDAGVAGVPDPVAGELPGAVVVLESGKNMTEKEVMDYVASQVSNAKRLRGGVRFVDEVPKGLTGKIDGRAIREILKKPVAKM

SEQ ID NO: 29 - protein, 548 aa, Unknown
  Hotaria unmunsana
MEMEKEENVVYGPLPFYPIEEGSAGIQLHKYMQQYAKLGAIAFSNALTGVDISYQEYFDITCRLAEAMKNYGMKQEGTIALCSENCEEFFMPVLAGLYIGVAVAPTNEIYTLRELNHSLGIAQPTIVFSSRKGLPKVLEVQKTVTCIKTIVILDSKVNFGGYDCMETFIKKHVELGFRPTSFVPIDVKNRKQHVALLMNSSGSTGLPKGVLITHEGTVTRFSHAKDPIYGNQVSPGTAILTVVPFHHGFGMFTTLGYFACGYRVVMLTKFDEELFLRTMQDYKCTSVILVPTLFAILNKSELIDKFDLSNLTEIASGGAPLAKEVGEAVARRFNLPGVRQGYGLTETTSAFIITPEGDDKPGASGKVVPLFKVKVIDLDTKKTLGVNRRGEICVKGPSLMLGYLNNPEATKETIDDEGWLHTGDIGYYDEDEHFFIVDRLKSLIKYKGYQVPPAELESVLLQHPNIFDAGVAGVPDSEAGELPGAVVVMEKGKTMTEKEIVDYVNSQVVNHKRLRGGVRFVDEVPKGLTGKIDAKVIREILKKPQAKM

SEQ ID NO: 30 - protein, 548 aa, Unknown
  Lampyroidea maculate
MEMEKEENVVYGPQPFYPIEKGSAGIQLHKYMHQYAQLGAIAFSNALTGVDISYQEYFDISCRLAEAMENYGMKPEGRIALCSENCEEFFIPVLAGLYIGVGVAPTNEIYTLRELNHTLGIAEPTIVFSSKKGLPKVLEVQKTVTCIKTIVILNSKVNFGGYDCVETFIKKNVELGFQPTSFKPIDVKNRKEHVALIMNSSGSTGLPKGVQITHEATVTRFSHAKDPIYGNQVSPGTAILTVVPFHHGFGMFTTLGYFACGYRIVMLTKFDEEIFLKTMQDYKCTSVILVPTLFGILNKSELIDKFDLSNLTEIASGGAPLAKEVGEAVARRFNLPGVRQGYGLTETTSAFIITPEGDDKPGASGKVVPLFKVKVIDLDTKKTLGANRRGEICVKGPSLMKGYINNPEATKEIIDEEGWMHTGDIGYYDEDEHFFIVDRLKSLIKYKGYQVPPAELESVLLQHPDIFDAGVAGVPDPEAGELPGAVVVMEKGKTMTEKEIVDYVNSQVVNHKRLRGGVRFVDEVPKGLTGKIDAKVIREILKKPQAKM

SEQ ID NO: 31 - protein, 552 aa, Photuris pennsylvanica
MSIENNILIGPPPYYPLEEGTAGEQLHRAISRYAAVPGTLAYTDVHTELEVTYKEFLDVTCRLAEAMKNYGLGLQHTISVCSENCVQFFMPICAALYVGVATAPTNDIYNERELYNSLSISQPTVVFTSRNSLQKILGVQSRLPIIKKIIILDGKKDYLGYQSMQSFMKEHVPANFNVSAFKPLSFDLDRVACIMNSSGSTGLPKGVPISHRNTIYRFSHCRDPVFGNQIIPDTTILCAVPFHHAFGTFTNLGYLICGFHVVLMYRFNEHLFLQTLQDYKCQSALLVPTVLAFLAKNPLVDKYDLSNLHEIASGGAPLSKEISEIAAKRFKLPGIRQGYGLTETTCAIVITAEGEFKLGAVGKVVPFYSLKVLDLNTGKKLGPNERGEICFKGPMIMKGYINNPEATRELIDEEGWIHSGDIGYFDEDGHVYIVDRLKSLIKYKGYQVPPAELEALLLQHPFIEDAGVAGVPDEVAGDLPGAVVVLKEGKSITEKEIQDYVAGQVTSSKKLRGGVEFVKEVPKGFTGKIDTRKIKEILIKAQKGKSKSKAKL

SEQ ID NO: 32 - protein, 549 aa, Unknown
  Lucidina biplagiata
MEEDKNILRGPAAFYPLEDGTAGEQLHRAMKRYALIPGTIAFTDAHAGVNITYSEYFEMACRLAESLKRYGLGLQHRIVVCSENSLQFFMPVVGALFIGVGVAPANDIYNERELLNSMTISQPTLVFCSRKGLQKILNVQKKLPVIQKIIILDTKEDYMGFQSMYSFVDSQLPVGFNEYDYVPDSFDRDQATALIMNSSGSTGLPKGVELTHTSVCVRFSHCRDPVFGNQIIPDTAILSVIPFHHGFGKFTTLGYLICGFRVVLMYRFEEELFLRSLQDYKIQSALLVPTLFSFFAKSTLIDKYDLSNLHEIASGGAPLAKEVGEAVAKRFNLRGIRQGYGLTETTSAVIITPEGDDKPGAVGKVVPFFSAKVVDLDTGKTLGVNQRGELCLKGPMIMKGYVNNPEATNALIDKDGWLHSGDISYWDEDGHFFIVDRLKSLIKYKGYQVPPAELESILLQHPFIFDAGVAGIPDDEAGELPAAVVVLEEGKTMTEKEIMDYVAGQVTTAKRLRGGVVFVDEVPKGLTGKLDARKIREILVKAKKTKSKL

SEQ ID NO: 33 - protein, 547 aa, Unknown
  Cratomorphus distinctus
MEEDKNIMYGPAPFSPLEEGTAGEQLHKAMKRYAQIPGTIAFTAAHVEVNVTYAEYFEMACRLAETMKRYGLGLDHRIAVCSENSLQFFMPVCGALFIGVGVAPTNDIYNERELYNSLSISQPTVVFCSKRALQKILGVQKSLPVIKKIVILDSREDYMGKQSMYSFIQSYLPGGFNEYDYVPDTFDRDMATALIMNSSGSTGLPKGVELSHKNVCVRFSHCRDPVFGNQIIPDTAILTVIPFHHGFGMFTTLGYLTCGFRIVLMYRFEEELFLRSLQDYKIQSALLVPTLFSFFAKSTLVDKYDLSNLHEIASGGAPLAKEVGEAVAKRFKLPGIRQGYGLTETTSAIIITPEGDDKPGACGKVVPFFAAKIVDLDTGKTLGVNQRGELYVKGPMIMKGYVNNPEATNALIDKDGWLRSGDIAYYDEDGHVFIVDRLKSLIKYKGYQVPPAELESILLQHPFIFDAGVAGIPDEDAGELPAAVVVLEEGKTMTEQEVMDYVAGQVTASKRLRGGVKFVDEVPKGLTGKIDSRKIREILVMGKKSKL

SEQ ID NO: 34 - protein, 547 aa, Unknown
  Diaphanes pectinealis
MEDDKNIIYGPPPFYPLEDGTAGEQLHRAMKRYAQVPGTIAFTDAHAEVNITYSEYFELSCRLAEAMKRYGLGLQHRIAVCSENSLQFFIPVCSALYIGAGVAPTNDIYNERELYNSLSISQPTIVFCSKRALQKILGVQKKLPVIEKIVILDSREDYMGKQSMYSFIDSHLPAGFNEYDYKPDSFDRDTATALIMNSSGSTGLPKGVDLSHKNVCVRFSHCRDPVFGNQIIPDTAILTVIPFHHGFGMFTTLGYLTCGFRVVLMYRFEEELFLRSLQDYKIQSALLVPTLFSFFAKSTLVDKYDLSNLHEIASGGAPLAKEVGEAVAKRFKLPGIRQGYGLTETTSAIIITPEGDDKPGACGKVVPFFCGKIVDLDTGKTLGVNQRGELCVKGPMIMKGYINNPEATNALIDKDGWLHSGDIAYYDKDGHFFIVDRLKSLIKYKGYQVPPAELESILLQHPFIFDAGAAGIPDPDAGELPAAVVVLEEGKTMTEQEVMDYVAGQVTASKRLRGGVKFVDEVPKGLTGKIDSRKIREILMMGKKSKL

SEQ ID NO: 35 - protein, 548 aa, Unknown
  Pyrocoelia pygidialis
MEDDHKNIMHGPAPFYPLEDGTAGEQLHKAMKRYAQVPGTIAFTDAHVEVNITYSEYFEMACRLAETMKRYGLGLQHHIAVCSENSLQFFMPVCGALFIGVGVAPTNDIYNERELYNSLSISQPTIVFCSKRALQKILGVQKKLPVIEKIVILDSREDYMGKQSMYSFIESHLPAGFNEYDYIPDSFDRDTATALIMNSSGSTGLPKGVELTHKNVCVRFSHCRDPVFGNQIIPDTAILTVIPFHHGFGMFTTLGYLTCGFRIVLMYRFEEELFLRSLQDYKIQSALLVPTLFSFFAKSTLVDKYDLSNLHEIASGGAPLAKEVGEAVAKRFKLPGIRQGYGLTETTSAIIITPEGDDKPGACGKVVPFFSAKIVDLDTSKTLGVNQRGELCVKGPMIMKGYVNNPEATSALIDKDGWLHSGDIAYYDKDGHFFIVDRLKSLIKYKGYQVPPAELESILLQHPFIFDAGVAGIPDPDAGELPAAVVVLEEGKTMTEQEVMDYVAGQVTASKRLRGGVKFVDEVPKGLTGKIDSRKIREILTMGKKSKL

SEQ ID NO: 36 - protein, 547 aa, Unknown
  Nyctophila cf. caucasica
MEDAKNIMHGPPPFYPLEDGTAGEQLHKAMKRYAQVPGTIAFTDAHAEVNITYSEYFEMACRLAETMKRYGLGLQHHIAVCSENSLQFFMPVCGALFIGVGVAPTNDIYNERELYNSLSISQPTIVFCSKRALQKILGVQKKLPIIQKIVILDSREDYMGKQSMYSFIESHLPAGFNEYDYIPDSFDRETATALIMNSSGSTGLPKGVELTHKNICVRFSHCRDPVFGNQIIPDTAILTVIPFHHGFGMFTTLGYLTCGFRIVLMYRFEEELFLRSLQDYKIQSALLVPTLFSFFAKSTLVDKYDLSNLHEIASGGAPLAKEVGEAVAKRFKLPGIRQGYGLTETTSAIIITPEGDDKPGACGKVVPFFSAKIVDLDTGKTLGVNQRGELCVKGPMIMKGYVNNPEATSALIDKDGWLHSGDIAYYDKDGHFFIVDRLKSLIKYKGYQVPPAELESILLQHPFIFDAGVAGIPDPDAGELPAAVVVLEEGKTMTEQEVMDYVAGQVTASKRLRGGVKFVDEVPKGLTGKIDARKIREILMMGKKSKL

SEQ ID NO: 37 - protein, 547 aa, Lampyris noctiluca
MEDAKNIMHGPAPFYPLEDGTAGEQLHKAMKRYAQVPGTIAFTDAHAEVNITYSEYFEMACRLAETMKRYGLGLQHHIAVCSENSLQFFMPVCGALFIGVGVAPTNDIYNERELYNSLSISQPTIVFCSKRALQKILGVQKKLPIIQKIVILDSREDYMGKQSMYSFIESHLPAGFNEYDYIPDSFDRETATALVMNSSGSTGLPRGVELTHQNVCVRFSHCRDPVFGNQIIPDTAILTVIPFHHGFGMFTTLGYLTCGFRIVLMYRFEEELFLRSLQDYKIQSALLVPTLFSFFAKSTLVDKYDLSNLHEIASGGAPLAKEVGEAVAKRFKLPGIRQGYGLTETTSAIIITPEGDDKPGACGKVVPFFSAKIVDLDTGKTLGVNQRGELCVKGPMIMKGYVNNPEATSALIDKDGWLHSGDIAYYDKDGHFFIVDRLKSLIKYKGYQVPPAELESILLQHPFIFDAGVAGIPDPDAGELPAAVVVLEEGKTMTEQEVMDYVAGQVTASKRLRGGVKFVDEVPKGLTGKIDGRKIREILMMGKKSKL

SEQ ID NO: 38 - protein, 555 aa, Unknown
  Stenocladius azumai
MPSSMMSKKDLEDKNVVHGPDPYYLVDEGNAGQQLHKTILRYAQLPDTIAFTDGHTKRDVTYAHYFDLTCRLAESLKRYGLNLQSRIAVCSENNVEFFIPVVASLYLGVGVAPTNDIYNETELFNSLNISQPTIVFVSKRALHKILEVKKRIPIIKTVVVLDTEEDFMGYHCLHSFMKHYLPPNFDIMSYKPEEFARDGQLALIMNSSGSTGLPKGVMLAHRSVVVRFSHCKDPVFGNQIIPDTAILTVIPFHHGFGMFTTLGYLTCGFRIVLLRKFDEHYFLKCLQDYKIQSALLVPTLFSFFAKSTLVDQYDLSNLKEIASGGAPLAKEVGEAVAKRFKLPGIRQGYGLTETTSAVIITPEGEDKPGSTGKVVPFFSAKIVDLNNGKSVGPHQRGELCLKGDMIMMGYCNNKAATDEMIDKDGWLHSGDIAYYDEDGHFFIVDRLKSLVKYKGYQVAPAELEAVLLQHPCVFDAGVTGVPDDVAGELPGACVVLEKGKHVTEQEVMDYVAGQLSSYKKLRGGVRFIDEIPKGLTGKIDRKALKEILKKPQSKM

SEQ ID NO: 39 - protein, 545 aa, Unknown
  Phausis reticulata
MEDKNIVHGPPPVYPVDDGTAGQQLHKTILRYAKLPGTIAFTNAHTEVNVTYSEYFDMTCRLAEAMKRFGLDLKHNIAVCSENSLEFFVPVVAALYLGVGVAPTNDIYNERELFNSLNISQPTIVFSSKRAVHKIMEVRKQLPVIKKIIILDTKDDFMGMHSMHSFIKAHIPSSFNEMDFKPESFDRSDVVALIMNSSGSTGLPKGVALSHKNVVVRFSHCKDPVFGNQIVPDTAILTVIPFHHGFGMFTTLGYLTCGFRIVLMNKFEEELFLRSLQDYKIQSALLVPTLFSFFAKSTLVDKYDLSHLEEIASGGAPLAKEVGEAVAKRFKLPGVRQGYGLTETTSACIITPEGDDKPGSTGKVVPFFSAKIIDLDTRKSLGPNQRGELCLKGDMIMKGYVNNPEATNALIDKDGWLHSGDIAYYDEDGHFFIVDRLKSLIKYKGYQVAPAELEAVLLQHPFIFDAGVTGIPDEVAGQLPAACVVVEKGKTLTEKEIMDYVAGQVSSSKRLRGGVRFVDEIPKGLTGKIDGRQLKEIFKKPQAKL

SEQ ID NO: 40 - protein, 547 aa, Unknown
  Cyphonocerus ruficollis
MEMEDKNVVRGPAPFYPVDDGTAGEQLHKTILRYAQLSGTIAFTDAHTEVNVTYAEYYDATCRLAEAMKRYGLDLKHRIAVCSENSLEFFVPVIAALYLGVAVAPTNDIYNERELFNSLNISQPTIVFTSKRALHKILEVQKQLPVIKKIVVLDSKDDFMGMQSMHSFIKHYLPADFNELDYKPESFDRNEQVALIMNSSGSTGLPKGVQLTHKNVVVRFSHCKDPVFGNQIIPDTAILTVIPFHHGFGMFTTLGYVTCGFRIVVMHKFEEELFLRSLQDYKIQSALLVPTLFSFFAKSTLVDKYDLSNLHEIASGGAPLAKEVGEAVAKRFKLPGVRQGYGLTETTSAVIITPEGDDKPGSTGKIVPFFSAKILDLDTKKSLGPNQRGELCLKGDMIMKGYVNNPEATNALIDKDGWLHSGDIAYYDEDGHFFIVDRLKSLIKYKGYQVPPAELESVLLQHPYIFDAGVAGVPDEDAGELPGACVVLEKGKHLTAKEVMDYVAGQVSSYKRLRGGVRFIDEIPKGLTGKIDGKALREILKKPQAKL

SEQ ID NO: 41 - protein, 547 aa, Unknown
  Drilaster axillaris
MNMEDKNVVHGPASFYPVDDGTAGQQLHKTILRYAQLPGTIAFTDAHTEQNLTYAEYFDLTCRLAESMKRYGLGLKHAIAVCSENSLHFFAPVIAALYLGVAVAPTNDIYNERELYNSLNISQPTIVFCSKRALLKIMEVQKKLPVIQKIIVLDHKEDFQGLHSMHSFIKHYLPSHFNEHDFKPEKFDRNDQVALIMNSSGSTGLPKGVMLTHKNVVVRFSHCKDPVFGNQIIPDTAILTVIPFHHGFGMFTTLGYLTCGFRIVVMHKFEEELFLRSLQDYKIQSALLVPTLFSFFAKSTLVDKYDLSNLKEIASGGAPLAKEVGEAVAKRFKLPGVRQGYGLTETTSAVIITPEGEDKAGSTGKVVPFFSAKIIDLDTRQSLGPHQKGELCLKGDMIMKGYANNPEATNALIDKDGWLHSGDIAYYDEDGHFFIVDRLKSLIKYKGYQVAPAELESVLLQHPYIFDAGVTGIPDEVAGELPAACVVLEKGKHLTEKEVMDYVASQVSSSKKLRGGVRFIDEIPKGLTGKIDSRGLKEILKRPQSKM

SEQ ID NO: 42 - protein, 551 aa, Anopheles darlingi
MNPTHPLPTDEKFIISGPPEPANITESCSSLGMFLYKRLLRNGDTTALIDGVYSTELQYLELLEKAIRLAESLRRHANLQAGDVVGIVSENRLEFPVALYAAFFLGAAVAPINLTYTERELNHALNLSKPKVLFVSPYSAERVVKVAGENRNFIERVFLFGDENAFGSNVTLYEQFLATTSTINPHTFPILPTNVDEHVALILCSSGTTGLPKGVQLTQRNVMASVSLLSELEASFEVPVVVLGVIPWFHAFGCLTLINVLSNKLKLVSLPKFEEGLFLSCIENYRCSFVFVVPPLMVFLAKHPLVDSYDLSCVNTLLCGAAPLSRETEELVKRRIGVKHVLQGYGMSETTLATLVQTAECNKPGSVGKLQVGTRAKVIDLETGKPLGPNRPGELCFQGTQIMKGYIGNDKATRETIDQNGWLHTGDIGYYDEDYEFFIIDRLKELIKYKGYQVPPAEIESILLTNVKIKDAGVVGLPDELAGELPLAFVVKQPNVTLTEEEVRQYVAERTSPAKRLHGGVRFVEEIPKNVSGKILRRELRALLKKPHAKL

SEQ ID NO: 43 - protein, 555 aa, Unknown
  Culex quinquefasciatus
MASKDESRFIISGGPEPQNITEGFGSMGAFVRNRMRRNGNDVAVIDGVYGTEVQYLELLEQSVRLAECLRTLAGIRVGDVIGIVSENRLEFPAVLFGALFVGATVAPINLTYSERELEHAFSLSKPKLIFVSPFSADRVVAAARRNRHIVQKVVLFGDENPFVEGQDQRDVVLFEEFQRPVTFVNPLNFYIPTVDIDQHVALIMCSSGTTGLPKGVQLTHANLLASIALLEESSNLMEPPPGGIVLLGVLPWFHAYGCMTLINVICNKQKLVSLPKFEEGLFLSCIENYRCTMIFVVPPLVVFLAKHPLVDSYDLSSIDTLLCGAAPLSKETEDLVKARLNVRHVRQGYGMSETTLATLVQNGECHKSGSVGKVQIGTLAKVIDPETGKLLGPNQHGELCFKGSQIMKGYIGNEKATRETIDQDGWLHTGDVGYYDEDFEFFIVDRLKELIKYKGFQVPPAEIEAILLTNPKVKDAAVIGLPDEAAGELPLAFVVKQDGVDISEAEIKKYVADRTSPAKRLHGGVRFIAEIPKNLSGKILRRELRAMLQTIKSKL

SEQ ID NO: 44 - protein, 542 aa, Unknown
  Bactrocera latifrons
MTTLLPGNIVYAGPITCTETHAYNSLGHYILEKYKSYGTQTVMIDAVTGQEYTAKYMHDSVVRMAHVLQHLGIKAGDVVGLSSENNVRFAITMYAAFAINATVAPLNITYSEREVHHAINLSKPKVIFASKLTVNVIEKVAKNNSFVKSIVVLDPCSSNDKILNFDELMKSKKIASKDYFDTAPANKKDDVALIICSSGTTGLPKGVQLTQYNILFTLDSNLEPTSIPMGEITLLTVIPWFHAFGCITLITTTVLGTTLVYLPKFEENLFLGAIQKYRVLMAFMVPPLMVFLAKHPLVDKYDLSSLMVLLCGAAPLSKDTQDLITERIGVPIIRQGYGLSESTLSLLVQNDEACKPGSVGSIKLGIYGKVIDTETGRALGPNQRGELCFKGDCIMKGYIGDTKSTQSMIIDGWLHTGDIGYFDDDFEFFIVDRIKELIKYKGFQVPPAEIEALLLTNKKINDAAVIGKPDEESGELPLAFVVKQPNVELSEEEVIKFVAEHASPAKRLRGGVIFVNNIPKNLSGKILRRELRELLKNRRSKL

SEQ ID NO: 45 - protein, 520 aa, Blattella germanica
MDAHHILTGPEPSCPTPNTSLGRILYDSIKSHKNKVALVDAVTGETRTFSDILSKSEKLAESILSRGVKPGDVISICSENSIDFILPVLATYYVGATCAPLNPLYTTRELIHSINISKPSIMFCSQKALKTVEEMSHKVEFLKEIIVFGQSNSHSHTSFNSLLQNKSTNFQPIDVDPKEFIAAILCSSGTTGLPKGVMLTQHNILTVFGTIHEPRFGSLREDDIALGLLPMFHSYAFACQLMTTYIGATVVVVNGFNEEVFLKSIQNYKITLLFLVPPLVIFLAKAKIVDNFNLSSIRSIRCGAAPLSEEVEVLLNERLGVGPVLQGYGMTETTLAVLVTPTDKHKTGSSGVVVPGMKCKVVDLETGEILGPNRKGELCFKGPLIMKGYCGNLQATSATFDKEGFLRTGDVGYYDEDGFFFIVDRVPPAEIEALLLTHPEIKDAGVVGIPDDIAGELPLAFVVKQPDSKVTKEEIIQYIAGQVSPQKRLHGGVEFVENIPKTASGKILRRDLKLMLKAKL

SEQ ID NO: 46 - protein, 501 aa, Unknown
  Habropoda laboriosa
MPEENILHGPPLPVMEFENLSLGQLILNQLSIHRFWVAQVNAYTGKIQTFKDILDISRKLAIALDKEGLKKGDKIAICSENNLEFCLAVCAAFFLGVTVCPLNPLYTERELKHALSISKPKYIFISPFGAKNICKVAPQLFWLPKLIMLTESTDKILPCIDSLISNTIISDNFHACSVDVNDHVAVILCSSGTTGLPKGVMLTDKNFLTVIRNLVALLPDRLNNNSTSLALLPFFHVYSFSVMLANLIFGSNCIILPRFDEKIFLHAIEKYKIQHLTVVPPLMVFLAKHPIVDKYNLSSIKEIWCGAASLSKEIAKMVENRLNITIKQAYGLTETTLAVLQAPDNSTKYGSVGTLVPGISAKVIPVNGDESSEPLGPNSVGELCFKGDLIMKGYCNNEEATAATIDKDRWLHSGDVGYYDEQGYFYIVDRLKELIKYKGYQVPPAELEAILLTCPGVNDAAVIGLPHEETGELPTAFVVKQEGSDVTAEEITNFVNGKFLN

SEQ ID NO: 47 - protein, 539 aa, Unknown
  Camponotus floridanus
MSKHAHILHGKGELVAEFNFSVGQLLLRQLTTHDTRIAQIDAHTGKEQTFKYILDTSRKLAIYLQKEGLTVNDGIAICSENNLDFCIPVCATFYLGAIVCPLNPLYSKGELKHALTIIKPKYIFISVIALNNMISIFKELHWFPRVLLLNNYDNIDIPWMSMDKAISNVSDDNIDKFQAVPVDINNHVTAILCSSGTTGLPKGVMWTDKNITTIIRMYINHPFVANAMSLALLPFFHAYSFVLLVIRLIGGNSSVVFSRFEEKLFLQSIEKYKIQYLTVVPPIMVFLAKHPLVDKYDLSSIRKIWCGAAPLSEKIEKAVVKRLNIPEISQGYGLTETTLAVLRFPQDTALKFGSVGMLVPGVSAKVIPLGEYETDETLGPNCEGELCFKGDLIMKGYYNDEKSTRATIDKDGWLHTGDVGYYDEEGYFFIVDRIKELIKYKGYQVPPAELEAILLTYSGIKDAAVIGIPNEEAGELPMAFIVKEENANIREEDIIQYVNERVSNPKRLRGGIKFVDSIPKTPSGKILRRVLRRTLKSKL

SEQ ID NO: 48 - protein, 540 aa, Unknown
  Acromyrmex echinatior
MSQQSNILRNEEELLPEFNISIGQCLLMQLTTHGTRVAHINPHTGKEQTFQYILDTSRRLAVYLQREGLKVNDTIAVCSENNLEFCIPVCAAFYLGAIACPLNPLYSEREFKHALSISKPKYIFISTVSFNGIRKIFRELHWSPKILMLTDGNNNVSWASMYKVMSNVSIDDANALQPASVNLDDHVTAILCSSGTTGLPKGVMLTDKNITTVIRMYMNTNTIPENAVSLSLLPFFHAYSFVFMILTILRGNCSIIFSHFEEELFLQYIEKYKIEYMPMVPSLMVFLAKHPLVDKYDLSCVKTIWSGAAPLSKEIQQAVAKRLNMNIIDVKQGYGLTETTLAVLRSPDGKGKLGSVGVVVPGTLVKVIPIGEYETDKALGPNCEGELCFKGDLIMKGYYNDEESTRATIDKDGWLHTGDVGYYDEDGYFYIVDRIKELIKYKGYQVPPAELEAILLTFPGVQDAAVIGIPNDKTGELPMAFIVKEENSNICEKDIIQYVNERVSNPKRLRGGIRFVDSIPKTPSGKILRRVLRDRLKSKL

SEQ ID NO: 49 - protein, 506 aa, Unknown
  Trachymyrmex zeteki
MPQQSNILRGEGELLPELKISMGQLLLMQLTTHGTRVAHINPHTGKEQTFQHILDTSRRLAVYLQREGLKANDTIAVCSENNLEFCIPVCAAFYLGAIACPLNPLYSERELKHALFISKPKYIFISMISFNGMRKVFRELHWSPKILMLTDGSNNVSWASMYKVISNVSNNDANALQAAPVNLDDHVTAILCSSGTTGLPKGVMLTDKNITTVISIFMNTNTIPENAVSLSLLPFFHAYSFVLMVLGLLKGNCSVVFSRFEEKLFLQYIEKYRIEYLPVVPSLMVFLAKHPLVDKYDLSCVKTIWSGAAPLSKEIQQAVAKRLNITSVKQGYGLTETTLAVLRSPDEKEKLGSVGVIVSGMSAKVIPIGEYETDEALGPNCEGELCFKGDLIMKGYYNDEKSTRAIIDKDGWLHTGDVGYYDEDGYFFIVDRIKELIKYKGYQVPPAELEAILLTCPGIQDAAVIGIPNDKTGELPMAFVVKEENSNICEKDIIQYVNGKNFQYHI

(19) United States
(12) Patent Application Publication
(10) Pub. No.: US 2023/0152329 A1
(43) Pub. Date: May 18, 2023

(54) NOVEL SPLIT-LUCIFERASE ENZYMES AND APPLICATIONS THEREOF

(71) Applicant: ARIZONA BOARD OF REGENTS ON BEHALF OF THE UNIVERSITY OF ARIZONA, Tucson, AZ (US)

(72) Inventors: Indraneel Ghosh, Tucson, AZ (US); Matthew Bienick, Tucson, AZ (US); Sean Campbell, Bronx, NY (US); Carl Buchholz, Roanoke, VA (US)

(21) Appl. No.: 17/905,675

(22) PCT Filed: Mar. 8, 2021
(86) PCT No.: PCT/US21/21378

§ 371 Date: Sep. 6, 2022

Related U.S. Application Data
(60) Provisional application No. 62/986,253, filed on Mar. 6, 2020.

(51) Int. Cl. G01N 33/68; C12Q 1/66

(52) U.S. Cl. CPC G01N 33/6845; C12Q 1/66

(57) ABSTRACT

A method to produce novel split-protein sensors are described herein. The method implements a sequence dissimilarity (SD) based design that can identify potential split-sites in proteins to generate a split-protein pair, and then employs structure guided mutagenesis of the fragmented protein interface to generate split-protein sensors. The sensors have a signal to background ratio >200 and can be readily used to monitor protein-protein interactions and their inhibition in cells.

### CROSS-REFERENCES TO RELATED APPLICATIONS

This application claims benefit of U.S. Provisional Application No. 62/986,253 filed Mar. 6, 2020, the specification(s) of which is/are incorporated herein in their entirety by reference.

### STATEMENT REGARDING FEDERALLY SPONSORED RESEARCH OR DEVELOPMENT

This invention was made with government support under Grant Nos. R01 GM115595 awarded by National Institutes of Health, and CHE1506091 awarded by NSF. The government has certain rights in the invention.

### REFERENCE TO A SEQUENCE LISTING

Applicant asserts that the information recorded in the form of an Annex C/ST.25 text file submitted under Rule 13ter.1(a), entitled UNIA_20_05_PCT_Sequencing_Listing_ST25, is identical to that forming part of the international application as filed. The content of the sequence listing is incorporated herein by reference in its entirety.

### BACKGROUND OF THE INVENTION

### Field of the Invention

Strategies to monitor protein-protein interactions (PPIs) in cells allows for biophysical characterization while providing assays for developing inhibitors. The present invention features a split-protein luciferase method for monitoring PPIs and their inhibitors. The split-luciferase enzyme has been developed that can be used for a variety of cell or in vitro assays.

### Background Art

Interactions between proteins play a vital role in all aspects of a cell from life to death. Development of general strategies to selectively evaluate protein-protein interactions (PPIs) in complex environments such as a cell can provide insight regarding the energetics of protein binding and cellular communication as well as provide facile assays for developing therapeutics. For measuring PPIs in eukaryotic cells, several techniques have been developed including the yeast two-hybrid system, the fluorescence resonance energy-transfer (FRET) system, and affinity purification techniques coupled to mass spectrometry (AP-MS). The two-hybrid system is effective but is naturally an indirect measurement in the nucleus and can be time intensive. FRET is a useful technique but the availability of fluorescent labels and autofluorescence can sometimes be limiting.

Another direct and sensitive approach for monitoring PPIs in lysates and cells is the split-protein reassembly or protein fragment complementation methods. Most split-protein methods rely on the fragmentation of a parent protein such that the individual halves cannot spontaneously assemble at micromolar concentrations, largely attributed to the entropic cost. However, the parent protein activity can be restored by fusing two interacting proteins (PPI pair) to the individual split-protein fragments. The split-protein reassembly systems act as direct reporters of the PPI pair, where the signal strength is correlated to the affinity of the PPI. Split-protein reporters have been designed whose catalytic function can be coupled to easily measured outputs, such as a change in absorbance, fluorescence or luminescence. Domains have been appended to detect a large range of biologically relevant molecules.

One of the most widely utilized classes of split-protein reporters are based on luciferase enzymes with a luminescent output that are particularly attractive due to their low background, and in some cases, their favorable tissue penetration. Several luciferase based split-protein reporters have been engineered from organisms such as Photinus pyralis (firefly), Renilla reniformis (sea pansy), Gaussia princeps (Gaussia), and most recently Oplophorus gracilirostris (NanoLuc). In the current art, existing split-luciferases have been largely designed by careful visual inspection of existing crystal or NMR structures and focusing on fragmenting at loops and then testing known interacting PPI pairs such as coiled-coils or using the chemically induced dimerizer (CID), rapamycin, for FKBP/FRB binding. As another example, a comprehensive screen of protein fragments using incremental truncation libraries was used in firefly luciferase fragmentation. However, these previous approaches for engineering split-proteins are time intensive, costly, and the resultant data sets could be noisy and have more false positives. Furthermore, these techniques may have missed other potentially useful fragmentation sites. Hence, there exists a need for new split-proteins and methods of designing them.

The present invention features a sequence dissimilarity method to predict potential new sites of fragmentation. The sequence dissimilarity approach was implemented on firefly-luciferase (Floc), which ultimately resulted in novel split-enzymes that can be utilized for monitoring PPIs.

### BRIEF SUMMARY OF THE INVENTION

Strategies to monitor protein-protein interactions (PPIs) in cells allow for biophysical characterization of the proteome in its native environment while providing convenient assays for developing inhibitors for PPIs. Split-protein complementation methods using luciferase, a class of oxidative enzymes that produce bioluminescence, have emerged as a facile approach for measuring PPIs in cells. It is an objective of the present invention to provide novel split-firefly luciferase (split-Fluc) sensors that can be used for measuring PPIs and methods of designing said sensors, as specified in the independent claims. Embodiments of the invention are given in the dependent claims. Embodiments of the present invention can be freely combined with each other if they are not mutually exclusive.

In some aspects, the present invention features a two-step methodology that first uses sequence dissimilarity (SD) based design that can identify potential split-sites in proteins to generate several new first-generation split-Flues with signal to background of >20-fold. In an exemplary embodiment, this approach identified several novel split-sites in firefly luciferase. The method then employs structure guided mutagenesis of the fragmented enzyme interface to generate second-generation split-Floc PPI sensors with signal to background of >200. Thus, the present invention also provides new split-Floc sensors that can be readily used to monitor PPIs and their inhibition in mammalian cells.

Without wishing to limit the invention to any theory or mechanism, it is believed that these technical features of the present invention advantageously provide split-Flue PPI sensors with high sensitivity and low background, and that are less time intensive to make. In some embodiments, the invention may be utilized to monitor molecular interactions, e.g. PPIs, both in vitro and in live cells, as well as provide convenient assays for developing inhibitors for PPIs. Said assays may be used separately or in conjunction with a variety of similar cell or in vitro assays. Since the present invention has a different composition of matter to an existing split-luciferase that is widely used, the two assays can potentially be used separately or simultaneously. This may allow for monitoring of orthogonal PPI's simultaneously. In further embodiments, the present invention may be used to screen inhibitors/drugs, for example, predicting a protein function of target protein and drug ability of molecules. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A is a schematic for making a split-protein sensor of the present invention.

FIG. 1B shows non-limiting embodiments of the split-protein sensor of the present invention.

FIG. 10 shows a firefly luciferase topology with identified split-sites highlighted in red. Loop region lengths are not to scale.

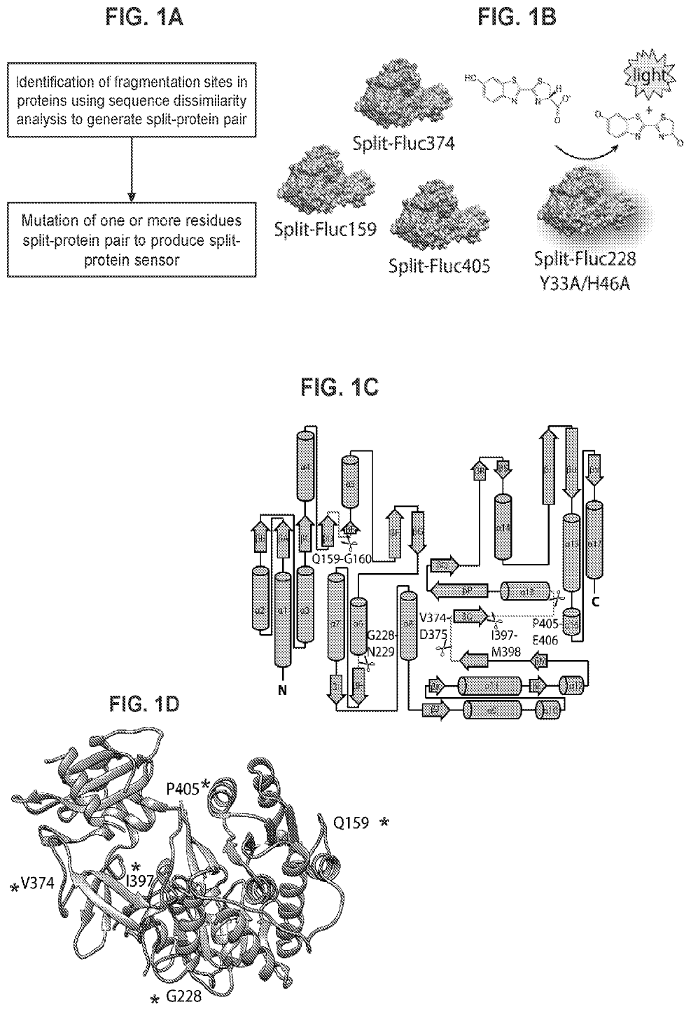

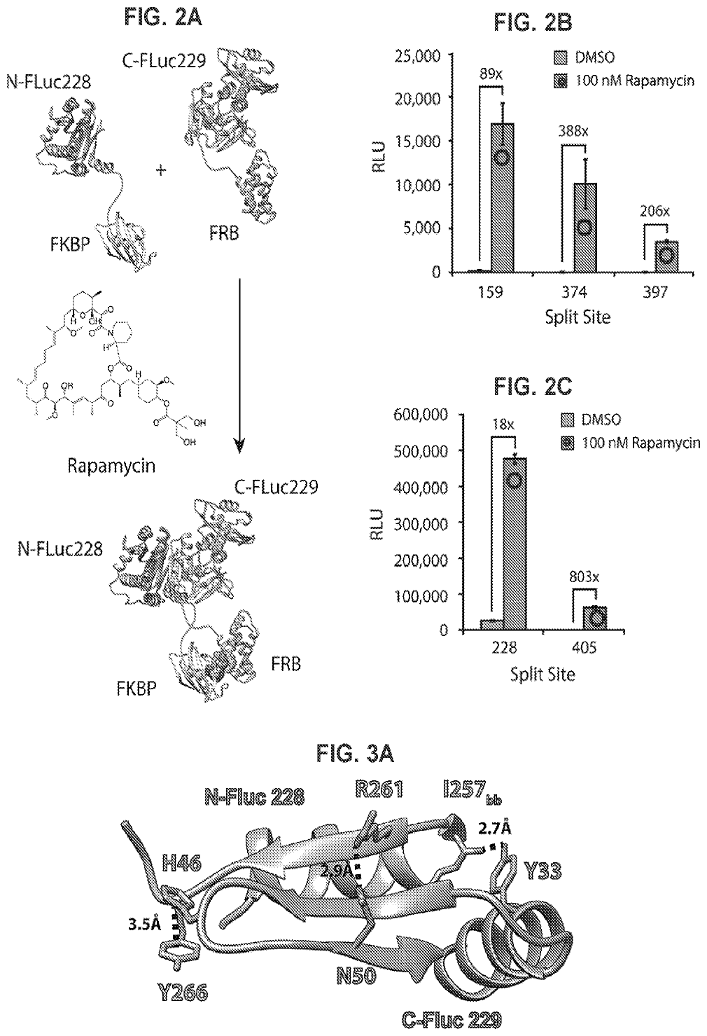

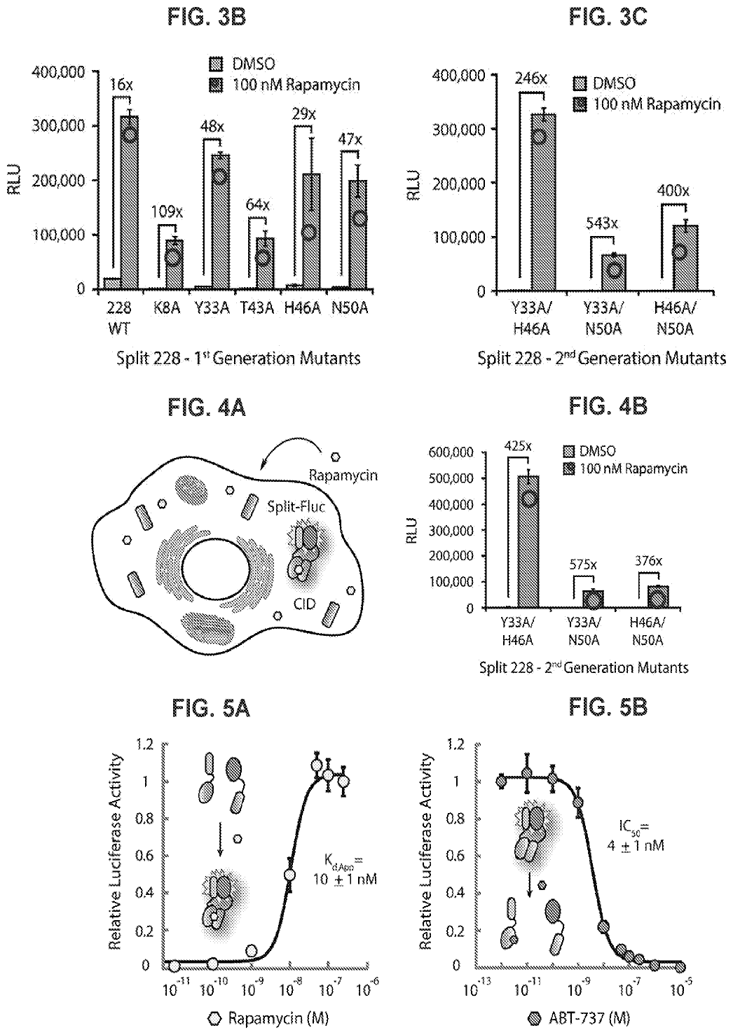

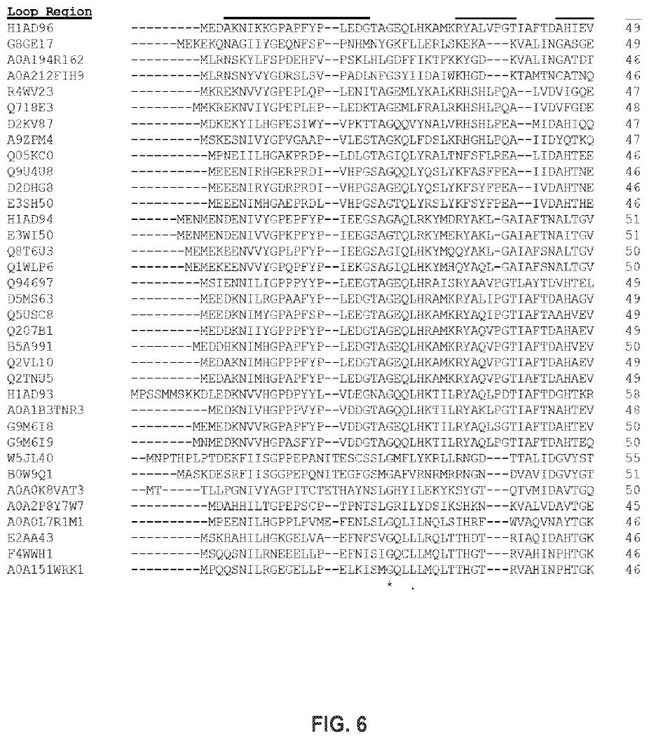

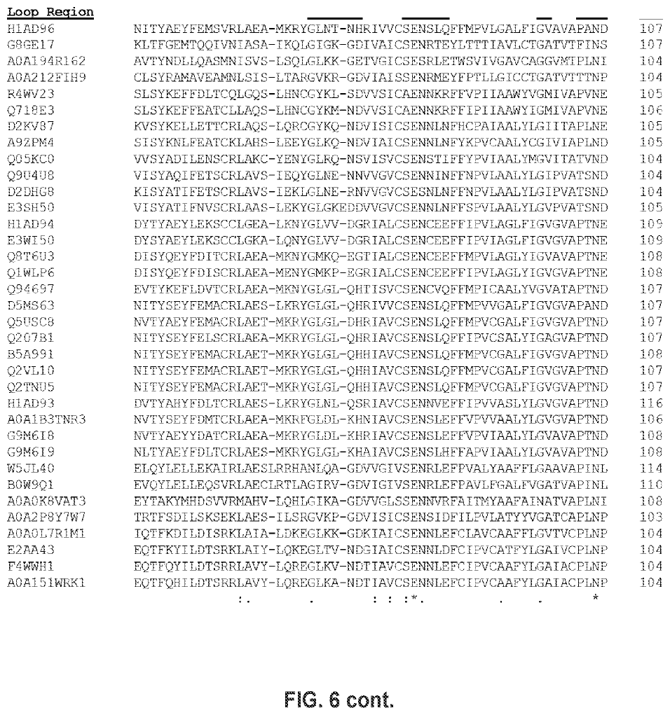

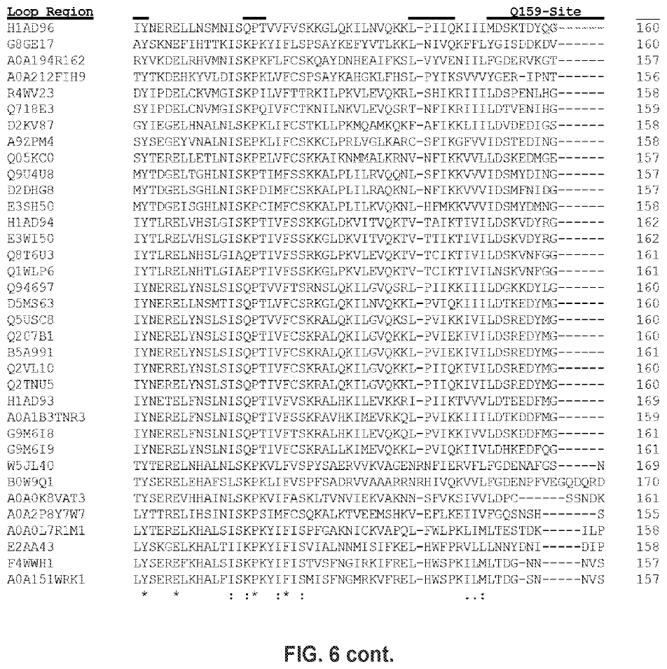

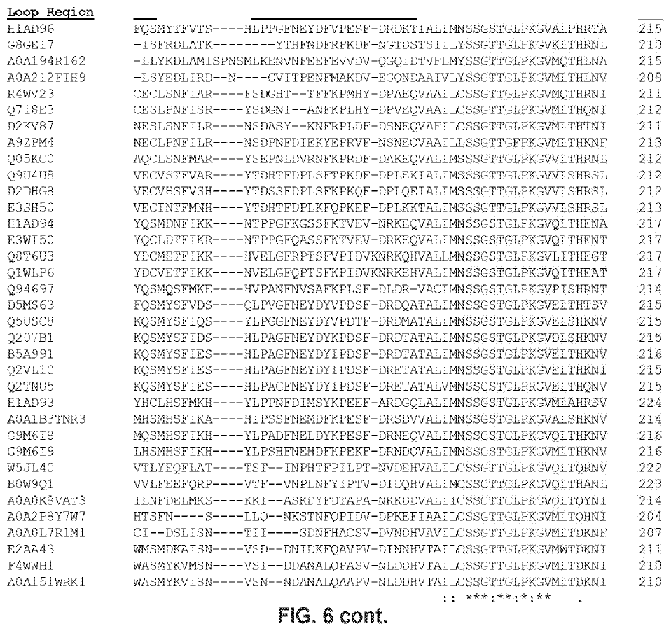

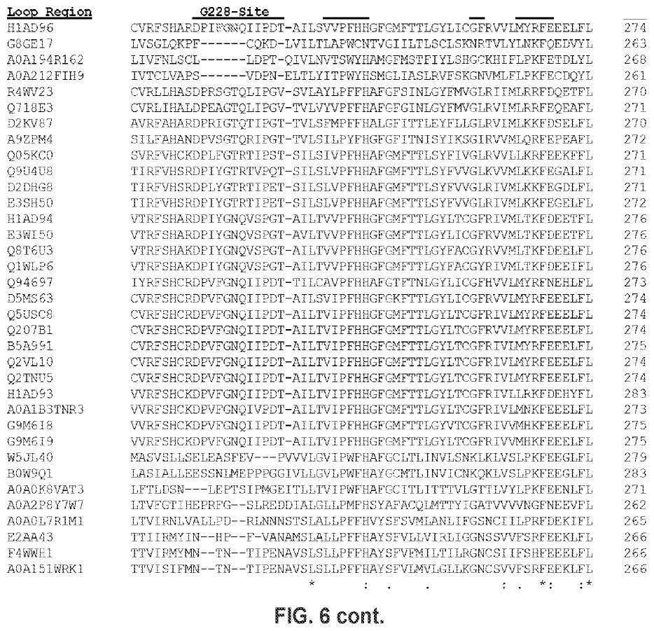

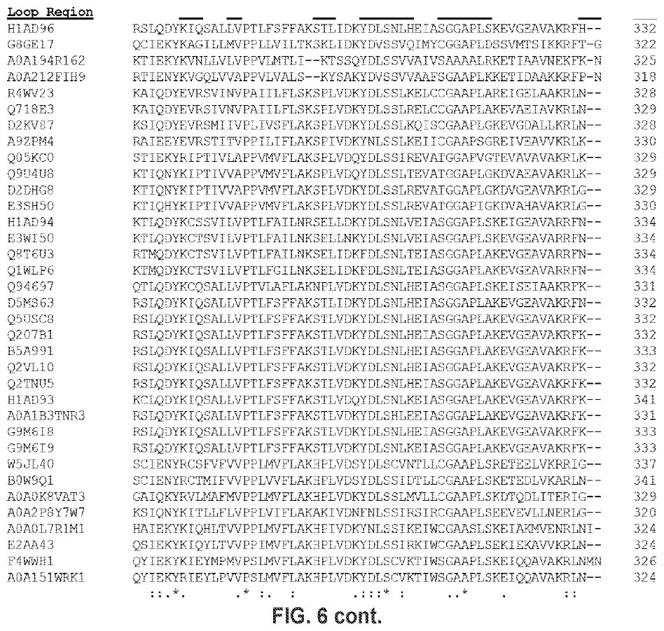

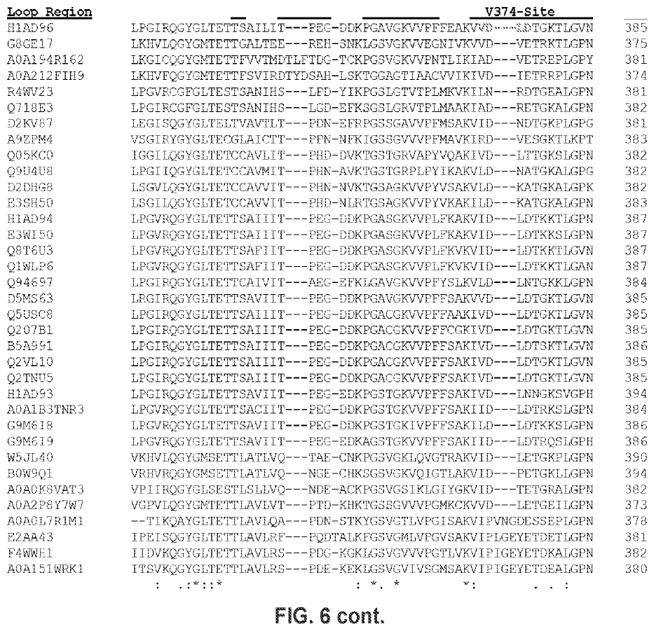

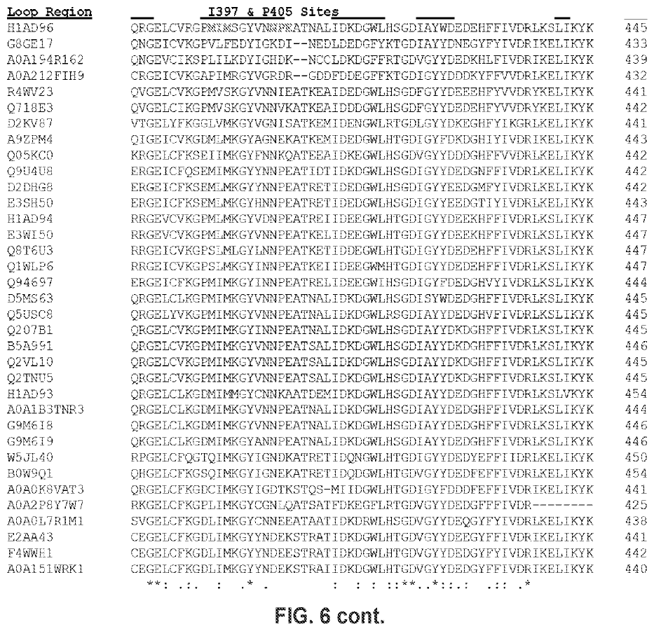

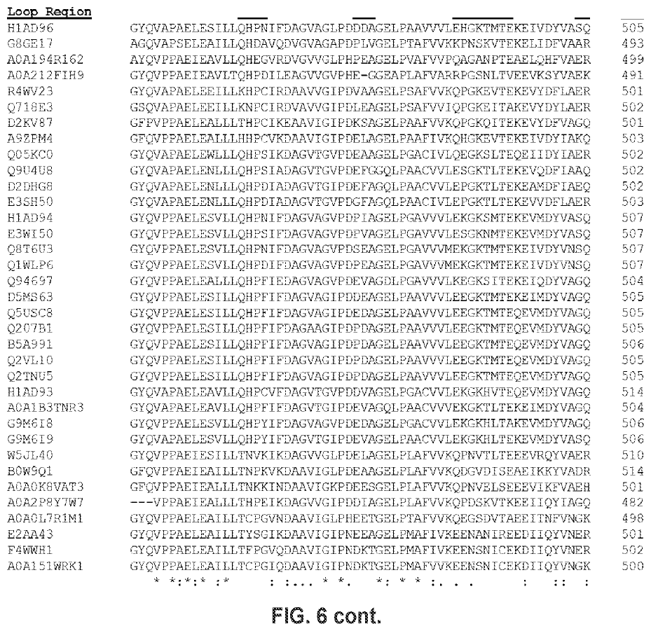

FIG. 1D shows a firefly luciferase crystal structure (PDB:1LCI) with loops used for split-sites denoted by a *.

FIG. 2A is a schematic of new split-luciferases. The FKBP domain was attached to the N-terminal half of Fluc by a 27-residue linker. The C-terminal half of Fluc was appended to the FRB domain by a 30-residue linker. Crystal structures—Luciferase PDB:1LCI, FKBP-Rap-FRB—PDB:1FAP.

FIGS. 2B and 2C are graphs of cell-free rapamycin inducible split-luciferase luminescence results of new split-firefly luciferases, Split-Fluc159, Split-Fluc374, and Split-Fluc397 (FIG. 2B), and Split-Fluc228 and Split-Flue405 (FIG. 2C) identified by sequence dissimilarity. Signal over background is shown for each set of experiments. Rapamycin is denoted by a circle.

FIG. 3A is a crystal structure representation of a select number split-firefly luciferase interactions targeted for mutagenesis. N-Fluc 228 and C-Fluc 229 are represented.

FIG. 3B show cell-free luciferase assay results of first-generation mutants of split-Fluc228 (K8A, Y33A, T43A, H46A, and N50A) compared to wild-type N-Fluc 228, which were treated with DMSO (control) or 100 nM rapamycin. The signal over background is shown for each set of experiments.

FIG. 3C shows cell-free luciferase assay results of second-generation mutants of split-Fluc228 (Y33A/H46A, Y33A/N50A, and H46A/N50A) treated with DMSO (control) or 100 nM rapamycin. The signal over background is shown for each set of experiments.

FIG. 4A illustrates split-Fluc under the control of a FKBP-Rapamycin-FRB system in cells.

FIG. 4B shows luminescence results of second-generation split-Fluc 228 mutants expressed in HEK293T cells followed by treatment with either 100 nM rapamycin or control (DMSO). The signal over background is shown for each set of experiments.

FIG. 5A shows the apparent K_{d} of the rapamycin induced interaction, FKBP-FRB, by split-Fluc228 Y33A/H46A luminescence.

FIG. 5B shows the IC₅₀ of ABT-737 against the protein-protein interaction of Bcl-xL and BAK determined by split-Fluc228 Y33A/H46A luminescence.

FIG. 6 shows CLustalO alignment of insect luciferases (SEQ ID NO: 15-49 respectively, in order of appearance) with chosen split-sites (fragment sites) of firefly Photinus pyralis luciferase (H1AD96) bolded (see Table 3 & 4). Loop regions of luciferase from Photinus pyralis luciferase (H1AD96) are underlined (See Table 3 & 4).

FIG. 7 shows alignment set 1 results from BLASTP of Photinus pyralis (H1AD96; SEQ ID NO: 15) vs. Photophorus jansonii (R4WV23; SEQ ID NO: 19), ‘+’ conserved amino acids.

FIG. 8 shows alignment set 2 results from BLASTP of Photophorus jansonii (R4WV23; SEQ ID NO: 19) vs. Danaus plexippus plexippus (A0A212FIH9; SEQ ID NO: 18), ‘+’ conserved amino acids.

FIG. 9 shows alignment set 3 results from BLASTP of Photinus pyralis (H1AD96; SEQ ID NO: 15) vs. Danaus plexippus plexippus (A0A212F1H9; SEQ ID NO: 18), ‘+’ conserved amino acids.

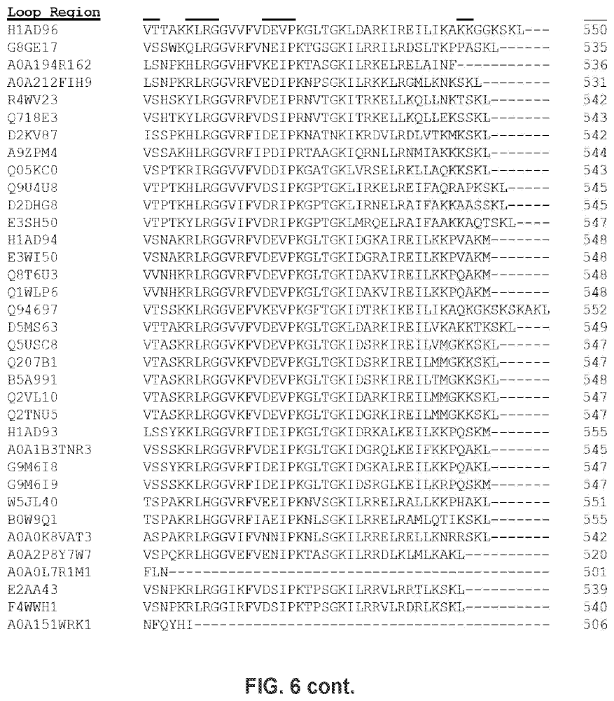

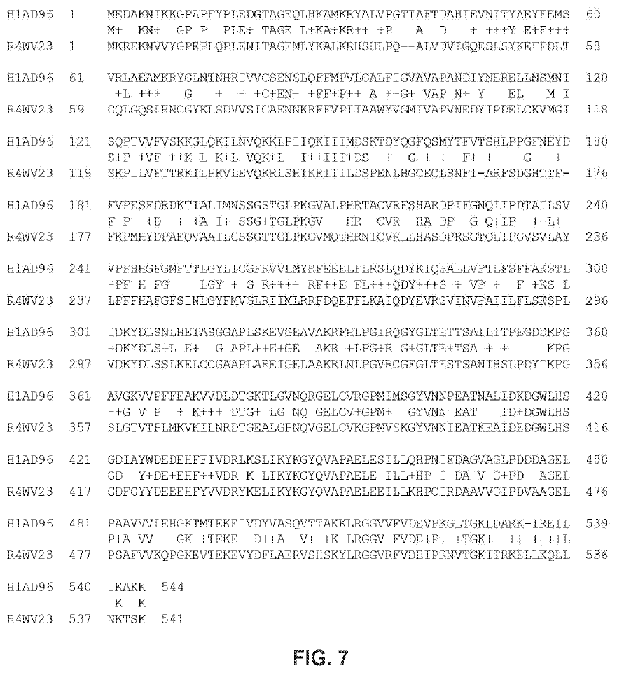

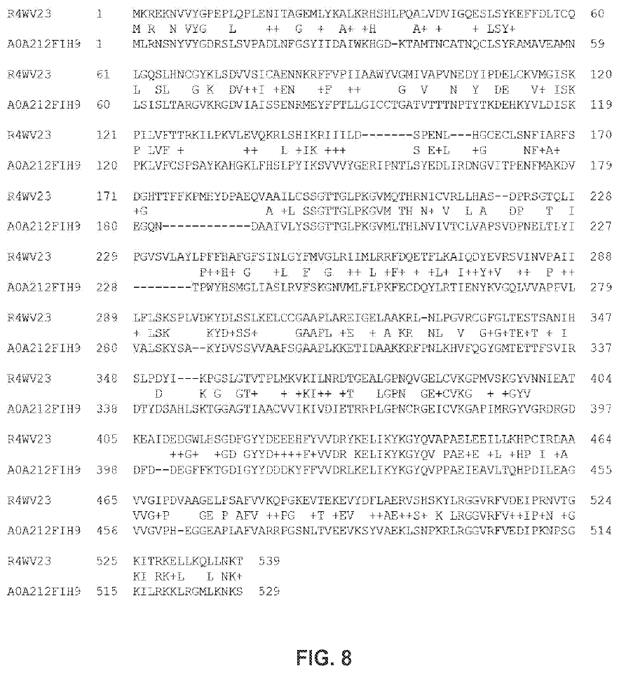

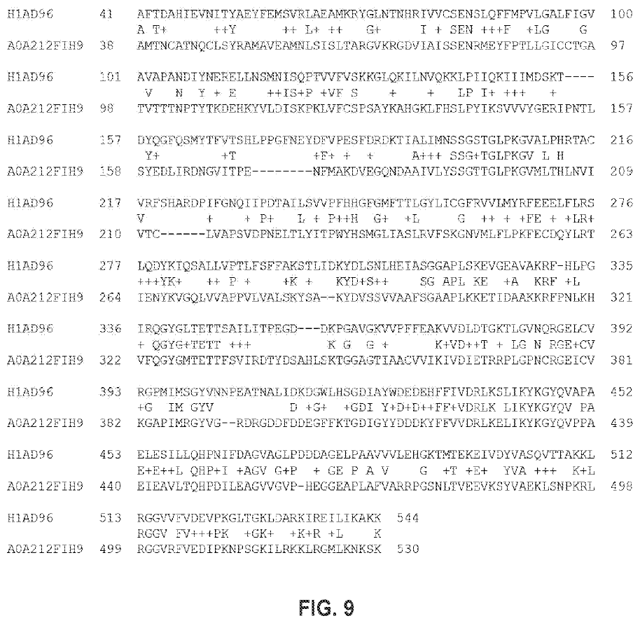

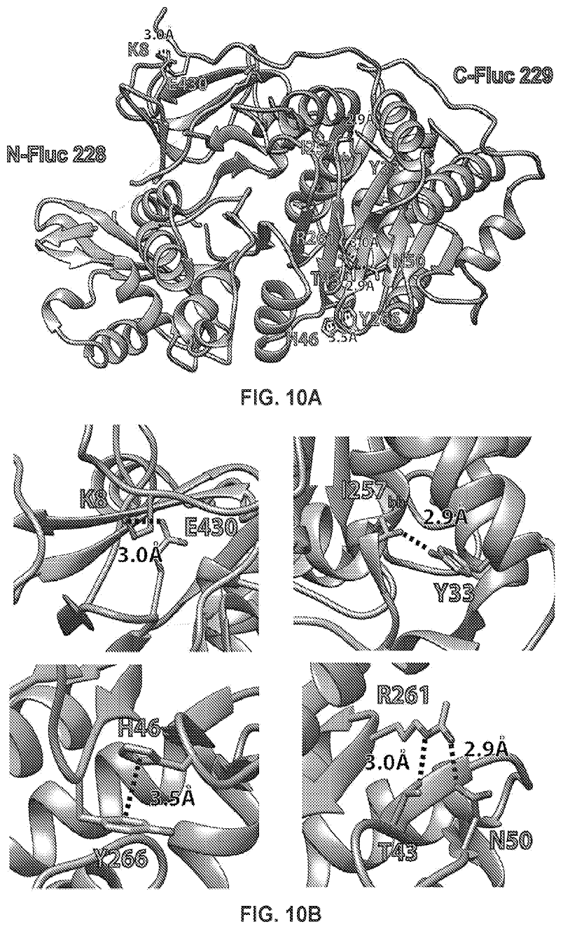

FIGS. 10A-10B show a crystal structure representation of split-Fluc228 interactions targeted for mutagenesis. All alanine mutations were done to the N-terminal fragment of split-Fluc228. N-Fluc228 and C-Fluc229 are represented. FIG. 10B is zoom-in views of the crystal structure representation in FIG. 10A.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Split-protein complementation provides a robust approach to monitoring protein-protein interactions (PPIs) and their inhibitors. Split-protein systems have also been used to monitor a range of biologically relevant macromolecules. Herein, the present invention provides a novel method of designing a new split-firefly luciferase (split-Fluc) both in vitro and in cells.

In some embodiments, the method implements a sequence dissimilarity-based design strategy that can be used for identifying potential split-sites in proteins that range from protein kinases and phosphatases to firefly luciferase by including sequences from related species. This dissimilarity-based prediction method resulted in four new split-Flucs. The present invention has demonstrated that the first generation split-Fluc sensors identified from the fragment sites can be significantly improved by mutagenesis. This step focused on mutating residues across the newly created split-protein interface that appeared likely to contribute to fragment binding through van der Waals or electrostatic stabilization. In one embodiment, mutation of the chosen residues to alanine was sufficient to considerably decrease unassisted reassembly of the fragments. Without wishing to limit the invention to a particular theory or mechanism, the sequence dissimilarity approach followed by interface optimization can generate user-defined split-proteins that have a wide-range of applications both in vitro and in cells. This overall two-step design strategy can produce novel split-Flucs that provide excellent protein-protein and small molecule induction of luminescence with over 200-fold over background complementation.

In one embodiment, the present invention features a useful and new split-Fluc228 Y33A/H46A sensor that can be used to monitor a variety of macromolecular interactions and their inhibition. In some embodiments, given that the new split-Fluc228 and existing split-Fluc have distinct fragments, it is unlikely that they can cross-complement and thus two different sets of interactions can be potentially monitored with the development of orthogonal substrates.

Referring now to FIGS. 1A-10B, the present invention features compositions and methods for monitoring protein-protein interactions (PPI) in cells which allows for biophysical characterization and development of inhibitors. The compositions and method described herein features a split-protein luciferase for monitoring PPIs and their inhibitors.

The present invention features a method of preparing a split signal-generating protein. In some embodiments, the method comprises identifying a fragmentation site in a signal-generating protein using sequence dissimilarity analysis. In other embodiments, the method comprises splitting the signal-generated protein at the fragmentation site to produce a first protein fragment and a second protein fragment. In further embodiments, the method comprises mutating at least one residue in one or both of the protein fragments.

In present invention may also feature a method of producing a split-protein sensor for detecting protein-protein interactions (PPI). In some embodiments, the method comprises analyzing a signal-generating protein using sequence dissimilarity to identify a fragmentation site in said protein. In other embodiments, the method comprises splitting the signal-generating protein at the fragmentation site to produce a first protein fragment and a second protein fragment. In some embodiments, the method comprises mutating at least one residue in one or both of the protein fragments. In further embodiments, the method comprises connecting the first protein fragment to a first protein of the protein-protein interaction via a first linker to form a first sensor complex. In other embodiments, the method comprises connecting the second protein fragment to a second protein of the protein-protein interaction via a second linker to form a second sensor complex. In some embodiments, the split-protein sensor comprises the first and second sensor complexes.

In some embodiments, the split signal-generating protein of the present invention is used in a split-protein sensor for detecting protein-protein interactions. In some embodiments, the split-protein sensor may comprise a first sensor complex comprising a first protein of the protein-protein interaction linked to the first protein fragment of the split signal-generating protein, and a second sensor complex comprising a second protein of the protein-protein interaction linked to the second protein fragment of the split signal-generating protein. In other embodiments, when the first and second proteins interact, said interaction causes the protein fragments to associate with one another and the signal-generating protein is re-assembled and exhibits a detectable signal.

As used herein a “signal-generating protein” may refer to a luciferase protein or a kztwjxhjsy uwtyjns. In some embodiments, the signal-generating protein may comprise a luminescent protein.

In some embodiments, the first linker or the second linker may comprise 27 amino acid residues. In other embodiments, the first linker or the second linker may comprise about 3-10 amino acid residues. In other embodiments, the first linker or the second linker may comprise about 9-20 amino acid residues. In other embodiments, the first linker or the second linker may comprise about 19-30 amino acid residues. In other embodiments, the first linker or the second linker may comprise about 29-40 amino acid residues. In other embodiments, the first linker or the second linker may comprise about 3-40 amino acid residues, or any value in between. In other embodiments, the linker is a serine-glycine linker. In other embodiments, other linkers with different amino acid compositions may be used at various lengths.

Table 1: Shows non-limiting examples of sequences derived from the Photinus pyralis luciferase gene. Bolded sequences indicate non-limiting mutations that may be made to the sequence. Underlined sequences indicated amino acids that were sequenced aligned to determine a potential fragment site/split site (see Table 2).

| | | SEQ ID |
|---|---|---|
| | Sequence | NO: |
| N-FLuc228 | MEDAKNIKKGPAPFYPLEDGTAGEQLHKAMKRYALVPGTIAFTDAHIEV | 1 |
| | NITYAEYFEMSVRLAEAMKRYGLNTNHRIVVCSENSLQFFMPVLGALFI | |
| | GVAVAPANDIYNERELLNSMNISQPTVVFVSKKGLQKILNVQKKLPIIQ | |
| | KIIIMDSKTDYQGFQSMYTFVTSHLPPGFNEYDFVPESFDRDKTIALIM | |
| | NSSGSTGLPKGVALPHRTACVRFSHARDPIFG | |
| C-FLuc229 | NQIIPDTAILSVVPFHHGFGMFTTLGYLICGFRVVLMYRFEEELFLRSL | 2 |
| | QDYKIQSALLVPTLFSFFAKSTLIDKYDLSNLHEIASGGAPLSKEVGEA | |
| | VAKRFHLPGIRQGYGLTETTSAILITPEGDDKPGAVGKVVPFFEAKVVD | |
| | LDTGKTLGVNQRGELCVRGPMIMSGYVNNPEATNALIDKDGWLHSGDIA | |
| | YWDEDEHFFIVDRLKSLIKYKGYQVAPAELESILLQHPNIFDAGVAGLP | |
| | DDDAGELPAAVVVLEHGKTMTEKEIVDYVASQVTTAKKLRGGVVFVDEV | |
| | PKGLTGKLDARKIREILIKAKKGGKSKL | |
| C-FLuc229 | AATCAAATCATTCCGGATACTGCGATTTTAAGTGTTGTTCCATTCCATC | 3 |
| (DNA) | ACGGTTTTGGAATGTTTACTACACTCGGATATTTGATATGTGGATTTCG | |
| | AGTCGTCTTAATGTATAGATTTGAAGAAGAGCTGTTTTTACGATCCCTT | |
| | CAGGATTACAAAATTCAAAGTGCGTTGCTAGTACCAACCCTATTTTCAT | |
| | TCTTCGCCAAAAGCACTCTGATTGACAAATACGATTTATCTAATTTACA | |
| | CGAAATTGCTTCTGGGGGCGCACCTCTTTCGAAAGAAGTCGGGGAAGCG | |
| | GTTGCAAAACGCTTCCATCTTCCAGGGATACGACAAGGATATGGGCTCA | |
| | CTGAGACTACATCAGCTATTCTGATTACACCCGAGGGGGATGATAAACC | |
| | GGGCGCGGTCGGTAAAGTTGTTCCATTTTTTGAAGCGAAGGTTGTGGAT | |
| | CTGGATACCGGGAAAACGCTGGGCGTTAATCAGAGAGGCGAATTATGTG | |
| | TCAGAGGACCTATGATTATGTCCGGTTATGTAAACAATCCGGAAGCGAC | |
| | CAACGCCTTGATTGACAAGGATGGATGGCTACATTCTGGAGACATAGCT | |
| | TACTGGGACGAAGACGAACACTTCTTCATAGTTGACCGCTTGAAGTCTT | |
| | TAATTAAATACAAAGGATATCAGGTGGCCCCCGCTGAATTGGAATCGAT | |
| | ATTGTTACAACACCCCAACATCTTCGACGCGGGCGTGGCAGGTCTTCCC | |
| | GACGATGACGCCGGTGAACTTCCCGCCGCCGTTGTTGTTTTGGAGCACG | |
| | GAAAGACGATGACGGAAAAAGAGATCGTGGATTACGTCGCCAGTCAAGT | |
| | AACAACCGCGAAAAAGTTGCGCGGAGGAGTTGTGTTTGTGGACGAAGTA | |
| | CCGAAAGGTCTTACCGGAAAACTCGACGCAAGAAAAATCAGAGAGATCC | |
| | TCATAAAGGCCAAGAAGGGCGGAAAGTCCAAATTG | |
| N-FLuc228 | MEDAKNIKKGPAPFYPLEDGTAGEQLHKAMKRAALVPGTIAFTDAAIEV | 4 |
| Y33A/H46A | NITYAEYFEMSVRLAEAMKRYGLNTNHRIVVCSENSLQFFMPVLGALFI | |
| (double | GVAVAPANDIYNERELLNSMNISQPTVVFVSKKGLQKILNVQKKLPIIQ | |
| mutant) | KIIIMDSKTDYQGFQSMYTFVTSHLPPGFNEYDFVPESFDRDKTIALIM | |
| | NSSGSTGLPKGVALPHRTACVRFSHARDPIFG | |
| N-FLuc228 | ATGGAAGACGCCAAAAACATAAAGAAAGGCCCGGCGCCATTCTATCCTC | 5 |
| Y33A/H46A | TAGAGGATGGAACCGCTGGAGAGCAACTGCATAAGGCTATGAAGAGAGC | |
| (double | GGCCCTGGTTCCTGGAACAATTGCTTTTACAGATGCAGCGATCGAGGTG | |
| mutant) | AACATCACGTACGCGGAATACTTCGAAATGTCCGTTCGGTTGGCAGAAG | |
| (DNA) | CTATGAAACGATATGGGCTGAATACAAATCACAGAATCGTCGTATGCAG | |
| | TGAAAACTCTCTTCAATTCTTTATGCCGGTGTTGGGCGCGTTATTTATC | |
| | GGAGTTGCAGTTGCGCCCGCGAACGACATTTATAATGAACGTGAATTGC | |
| | TCAACAGTATGAACATTTCGCAGCCTACCGTAGTGTTTGTTTCCAAAAA | |
| | GGGGTTGCAAAAAATTTTGAACGTGCAAAAAAAATTACCAATAATCCAG | |
| | AAAATTATTATCATGGATTCTAAAACGGATTACCAGGGATTTCAGTCGA | |
| | TGTACACGTTCGTCACATCTCATCTACCTCCCGGTTTTAATGAATACGA | |
| | TTTTGTACCAGAGTCCTTTGATCGTGACAAAACAATTGCACTGATAATG | |
| | AATTCCTCTGGATCTACTGGGTTACCTAAGGGTGTGGCCCTTCCGCATA | |
| | GAACTGCCTGCGTCAGATTCTCGCATGCCAGAGATCCTATTTTTGGC | |
| Fragment | DPIFGNQIIPDT | 6 |
| site | | |
| N-FLuc228 | MEDAKNIKKGPAPFYPLEDGTAGEQLHKAMKRYALVPGTIAFTDAAIEV | 7 |
| H46A (single | NITYAEYFEMSVRLAEAMKRYGLNTNHRIVVCSENSLQFFMPVLGALFI | |
| mutant) | GVAVAPANDIYNERELLNSMNISQPTVVFVSKKGLQKILNVQKKLPIIQ | |
| | KIIIMDSKTDYQGFQSMYTFVTSHLPPGFNEYDFVPESFDRDKTIALIM | |
| | NSSGSTGLPKGVALPHRTACVRFSHARDPIFG | |
| N-FLuc228 | MEDAKNIKKGPAPFYPLEDGTAGEQLHKAMKRAALVPGTIAFTDAHIEV | 8 |
| Y33A (single | NITYAEYFEMSVRLAEAMKRYGLNTNHRIVVCSENSLQFFMPVLGALFI | |
| mutant) | GVAVAPANDIYNERELLNSMNISQPTVVFVSKKGLQKILNVQKKLPIIQ | |
| | KIIIMDSKTDYQGFQSMYTFVTSHLPPGFNEYDFVPESFDRDKTIALIM | |
| | NSSGSTGLPKGVALPHRTACVRFSHARDPIFG | |
| N-FLuc228 | RYALVPGT | 9 |
| Y33 non- | | |
| mutated | | |
| N-FLuc228 | AHIEV | 10 |
| H46 non- | | |
| mutated | | |

Table 2 shows a non-limiting example of amino acid sequence alignment of firefly luciferase (Photinus pyralis) from multiple insect species with predicted dissimilar/fragment-site depicted in bold.

| α6-βH Alignment | | SEQ ID |
|---|---|---|
| (228-site) | Sequence | NO: |
| Photinus | SGSTGLPKGVALPHRTACVRFSHAR | 11 |
| pyralis | DPIFGNQIIPDT-AILSVVPFHHG | |
| Rhagophthalmus | SSGTTGLPKGVVLTHRNLSVRFHCK | 12 |
| ohbai | DPLFGTRTIPST-SILSIVPFHH | |
| Campontus | SGTTGLPKGVMWTDKNITTIIRMYI | 13 |
| floridanus | N--HP--F-VANAMSLALLPFFHA | |
| Papilio | SGTTGLPKGVMQTHLNALIVFNLSC | 14 |
| machaon | L------LDPT-QIVLNVTSWYHA | |

In one embodiment, the signal-generating protein is a luminescent protein. The luminescent protein can exhibit a detectable luminescence signal. In a non-limiting embodiment, the luminescent protein is firefly Photinus pyralis luciferase (H1AD96). In this example, the first and second protein fragments of the split signal-generating protein are derived from splitting a fragmentation site between the residues GN of the sequence DPIFGNQIIPDT (SEQ ID NO: 6) in firefly Photinus pyralis luciferase (SEQ ID NO: 15). The first protein fragment can have a mutation at a Y residue of an alpha helix in Photinus pyralis luciferase comprising the sequence RYALVPGT (SEQ ID NO: 9) and at an H residue of another loop comprising the sequence AHIEV (SEQ ID NO: 10). In one embodiment, these Y and H residues are replaced with A. When the first and second proteins interact, the firefly Photinus pyralis luciferase is re-assembled and exhibits a detectable luminescence.

As used herein, a “fragment site” may refer to a stretch of non-homologous or different amino acids identified from primary sequence alignment of closely related species encoding a similar gene. This stretch of amino acids can also be mapped to a solvent accessible loop region within the crystal or NMR structure of the protein of interest. In some embodiments, the fragmentation site is determined by sequence dissimilarity. As used herein, “fragment site” or “split site” or “fragmentation site” may be used interchangeably.

As used herein, “sequence dissimilarity” may refer to a region within a protein's primary sequence that when aligned to a sequence of a closely related species that expresses a similar gene is non-homologous or different.

In some embodiments, the present invention utilizes luciferase sequences available from closely related species within the insect kingdom, however, the present invention is not limited to only the insect kingdom. In other embodiments, the strategies and methods described herein could be used to compare signal-generating proteins in other related species within a kingdom (e.g. comparing marine organisms that are related—such as comparing luciferase proteins in sea pansy (phylum Cnidaria) or comparing fluorescent proteins in jellyfish).

In some embodiments, the luminescent protein may comprise Photinus pyralis luciferase (H1AD96). In some embodiments, the fragmentation site of Photinus pyralis luciferase (SEQ ID NO: 15) is between residues GN of a sequence DPIFGNQIIPDT (SEQ ID NO: 6). In other embodiments, mutating at least one residue comprises replacing a tyrosine (Y) residue of an alpha helix in Photinus pyralis luciferase comprising a sequence RYALVPGT (SEQ ID NO: 9) with alanine (A). In further embodiments, mutating at least one residue comprises replacing a histidine (H) residue of a loop in Photinus pyralis luciferase comprising a sequence AHIEV (SEQ ID NO: 10) with an alanine (A).

In preferred embodiments, one or both of the protein fragments have at least one mutated residue. In other embodiments, one or both of the protein fragments have at least two mutated residues. In some embodiments, one or both of the protein fragments have at least three mutated residues. In some embodiments, one or both of the protein fragments have at least five mutated residues. In some embodiments, one or both of the protein fragments have at least ten mutated residues. In some embodiments, one or both of the protein fragments have more than ten mutated residues.

In some embodiments, mutating at least one residue comprises replacing said residue with another amino acid, for example alanine mutagenesis. Without wishing to be bound to a particular theory or mechanism, it is believed that the mutations can reduce affinity of the protein fragments towards one another. When the protein fragments do associate with one another, the signal-generating protein is re-assembled, which exhibits a detectable signal.

In some embodiments, the first protein fragment may comprise SEQ ID NO: 1, SEQ ID NO: 4, SEQ ID NO: 7 or SEQ ID NO: 8. In some embodiments, the first protein fragment has 100% sequence identity to SEQ ID NO: 1. In other embodiments, the first protein fragment has 99.5% sequence identity to SEQ ID NO: 1 or 99% sequence identity to SEQ ID NO: 1. In some embodiments, the first protein fragment has 98% sequence identity to SEQ ID NO: 1, or 97% sequence identity to SEQ ID NO: 1, or 96% sequence identity to SEQ ID NO: 1. In some embodiments, the first protein fragment has 95% sequence identity to SEQ ID NO: 1, or 90% sequence identity to SEQ ID NO: 1, or 85% sequence identity to SEQ ID NO: 1, or 80% sequence identity to SEQ ID NO: 1. In other embodiments, the first protein fragment has 75% sequence identity to SEQ ID NO: 1 or 70% sequence identity to SEQ ID NO: 1.

In some embodiments, the second protein fragment may comprise SEQ ID NO: 2. In some embodiments, the second protein fragment has 100% sequence identity to SEQ ID NO: 2. In other embodiments, the second protein fragment has 99.5% sequence identity to SEQ ID NO: 2 or 99% sequence identity to SEQ ID NO: 2. In some embodiments, the second protein fragment has 98% sequence identity to SEQ ID NO: 2, or 97% sequence identity to SEQ ID NO: 2, or 96% sequence identity to SEQ ID NO: 2. In some embodiments, the second protein fragment has 95% sequence identity to SEQ ID NO: 2 or 90% sequence identity to SEQ ID NO: 2. In other embodiments, the second protein fragment has 85% sequence identity to SEQ ID NO: 2 or 80% sequence identity to SEQ ID NO: 2. In some embodiments, the second protein fragment has 75% sequence identity to SEQ ID NO: 2 or 70% sequence identity to SEQ ID NO: 2.

In some embodiments, the first protein fragment or the second protein fragment has 100% sequence identity to the signal-generating protein fragment. In other embodiments, the first protein fragment or the second protein fragment has 99.5% sequence identity to the signal-generating protein fragment or 99% sequence identity to the signal-generating protein fragment. In some embodiments, the first protein fragment or the second protein fragment has 98% sequence identity to the signal-generating protein fragment, or 97% sequence identity to the signal-generating protein fragment, or 96% sequence identity to the signal-generating protein fragment. In some embodiments, the first protein fragment or second protein fragment have 95% sequence identity to the signal-generating protein fragment or 90% sequence identity to the signal-generating protein fragment. In some embodiments, the first protein fragment or the second protein fragment has 85% sequence identity to the signal-generating protein fragment or 80% sequence identity to the signal-generating protein fragment. In some embodiments, the first protein fragment or the second protein fragment has 75% sequence identity to the signal-generating protein fragment or 70% sequence identity to the signal-generating protein fragment. In some embodiments, a signal-generating protein fragment is generated when a signal-generating protein is split at a fragmentation site. In other embodiments, splitting the signal-generating protein at the fragmentation site produces a first protein fragment and a second protein fragment.

In some embodiments, a detectable signal may be detected on a luminometer plate readers, charge-coupled device (CCD) camera, photon imager, or bioluminescence imaging workstations.

In some embodiments, the signal-generating protein has a signal to background ratio that is greater than 200. In other embodiments, the split-protein sensor of the present invention has a signal to background ratio that is greater than 200.

In some embodiment, the signal to background ratio may vary (the signal to noise ratio could be lower or higher) depending on the instrument used for detection, stability of substrate, etc.

In some embodiments, the protein-protein interaction is caused by the first and second proteins binding to a target molecule. Non-limiting examples of the target molecule include a biological molecule, drug compound, or other chemical compound. Thus, in addition to monitoring protein-protein interactions, the split-protein sensor of the present invention may be used for drug/inhibitor screening.

According to other embodiments, the present invention provides a method preparing a split signal-generating protein. The method may comprise identifying a fragmentation site in the signal-generating protein using sequence dissimilarity analysis, splitting the signal-generating protein at the fragmentation site to produce a first protein fragment and a second protein fragment, and mutating at least one residue in one or both of the protein fragments.

The present invention may also feature the use of the split signal-generating protein to produce a split-protein sensor for detecting protein-protein interactions. The present invention may feature a split-protein sensor for detecting PPI. In some embodiments, the split-protein sensor may comprise a first sensor complex comprising a first protein of a protein-protein interaction linked to a first protein fragment. In some embodiments, the split-protein sensor may comprise a second sensor complex comprising a second protein of the protein-protein interaction linked to a second protein fragment. In other embodiments, the first and second protein fragments are derived from splitting of a fragmentation site of a signal-generating protein. In some embodiments, the fragmentation site is determined by sequence dissimilarity. In other embodiments, one or both of the protein fragments have at least one mutated residue. In further embodiments, interaction of the first and second proteins causes re-assembly of the signal-generating protein which exhibits a detectable signal.

The split-protein sensor may be produced by connecting the first protein fragment to a first protein of the protein-protein interaction via a first linker to form a first sensor complex, and connecting the second protein fragment to a second protein of the protein-protein interaction via a second linker to form a second sensor complex. The split-protein sensor comprises the first and second sensor complexes.

The present invention may also feature a split-protein sensor for detecting protein-protein interactions. In some embodiments, the split-protein sensor may comprise a first sensor complex comprising a first protein of a protein-protein interaction linked to a first protein fragment. In some embodiments, the split-protein sensor may comprise a second sensor complex comprising a second protein of the protein-protein interaction linked to a second protein fragment. In some embodiments, the first and second protein fragments are derived from splitting a fragmentation site between residues GN of a sequence DPIFGNQIIPDT (SEQ ID NO: 6) in firefly Photinus pyralis luciferase (H1AD96; SEQ ID NO: 15). In other embodiments, the first protein fragment has a mutation at a Y residue of an alpha helix in Photinus pyralis luciferase comprising the sequence RYALVPGT (SEQ ID NO: 9) and at an H residue of another loop comprising the sequence AHIEV (SEQ ID NO:10), wherein said Y and H residues are replaced with A. In further embodiments, the first and second protein fragments cause reassembly of the firefly Photinus pyralis luciferase, which exhibits a detectable luminescence.

In preferred embodiments, the signal-generating protein has a signal to background ratio that is greater than 200. In some embodiments, the signal-generating protein is a luminescent protein. In a non-limiting example, the luminescent protein is Photinus pyralis luciferase (H1AD96). In one embodiment, the fragmentation site of Photinus pyralis luciferase is between residues GN of a sequence DPIFGNQIIPDT (SEQ ID NO 6).

In some embodiments, mutating at least one residue comprises replacing said residue with another amino acid. For example, in one embodiment, a Y residue of an alpha helix in Photinus pyralis luciferase comprising the sequence RYALVPGT (SEQ ID NO: 9) is mutated by replacing said residue with A. In other embodiments, an H residue of a loop in Photinus pyralis luciferase comprising the sequence AHIEV (SEQ ID NO: 10) is mutated by replacing said residue with A. Thus, a Photinus pyralis luciferase fragment can have one or two mutations.

In some embodiments, the split-protein sensor may be used in a variety of cell or in vitro assays, such as detection of molecular interactions and screening for inhibitors/drugs.

The present invention provides a method of detecting protein-protein interactions. In some embodiments, the method may comprise preparing a split-protein sensor, according to the methods described above. For example, preparation of a split-protein sensor may comprise analyzing a signal-generating protein using sequence dissimilarity to identify a fragmentation site in said protein. In other embodiments, preparation of a split-protein sensor may comprise splitting the signal-generating protein at the fragmentation site to produce a first protein fragment and a second protein fragment and mutating at least one residue in one or both of the protein fragments. In other embodiments, preparation of a split-protein sensor may comprise connecting the first protein fragment to a first protein of the protein-protein interaction via a first linker to form a first sensor complex and connecting the second protein fragment to a second protein of the protein-protein interaction via a second linker to form a second sensor complex. In further embodiments, the split-protein sensor comprises the first and second sensor complexes. In other embodiments, the method may further comprise detecting a signal from the split-protein sensor when the first and second proteins interact. In other embodiments, the protein-protein interaction causes the first and second protein fragments to associate, thereby reassembling the signal-generating protein that exhibits a detectable signal.

Without wishing to be bound to a particular theory or mechanism, the protein-protein interaction causes the first and second protein fragments to associate, thereby reassembling the signal-generating protein that exhibits a detectable signal. In some embodiments, the protein-protein interaction is caused by the first and second proteins binding to a target molecule. Examples of the target molecule include, but are not limited to, biological molecules, a drug compound, or other chemical compound.

### EXAMPLE

The following is a non-limiting, demonstrative example of the present invention. It is to be understood that said example is not intended to limit the present invention in any way. Equivalents or substitutes are within the scope of the present invention.

Materials and Methods

Cloning and mRNA Generation

N-terminal halves of firefly luciferase (N-Fluc) constructs were generated by fusing FK506-binding protein (FKBP) or Bcl-xL 209 (CΔ24) to the C-terminal of N-Fluc with a 27-residue glycine-serine linker in pcDNA 3.1 via restriction cloning. C-terminal halves of firefly luciferase (C-Fluc) were created by fusing either FKBP rapamycin-binding (FRB) domain or the BH3-domain of BAK to the N-terminal of C-Fluc with a 30-residue glycine-serine linker in pcDNA 3.1 via restriction cloning. For in vitro assays, mRNA was generated using RiboMaX large scale RNA production system (T7) for 4 h at 30° C. and subsequently purified using G50-microcolumns.

Cell-Free Protein Expression and In Vitro Split-Luciferase Assay

Briefly, 0.5 pmol of mRNA corresponding to each split-luciferase pair was translated in a total volume of 25 μL of rabbit reticulocyte lysate (RRL) either with the relevant small molecule, Rapamycin or ABT-737 or DMSO as control. Following translation, 20 μL of lysate was added to 80 μL of L-Glo luciferase assay reagent, incubated at room temperature for 1 min and luminescence was subsequently read using a luminometer with 10 s integration time. All reported data are at least in duplicate and assays have been repeated on different days. Data from the reported luciferase assays were all normalized with background correction of equivalent treatment of RRL without added mRNA.

Mammalian Cell Culture and in Cellulo Split-Luciferase Assay

HEK293T cells were maintained in DMEM supplemented with 10% (v/v) FBS, 2.5 μg/mL Amphotericin B, 100 U/mL penicillin, and 100 μg/mL streptomycin, and in a 37° C. water-saturated incubator with 5% CO₂. For in cellulo experiments, 3×10⁶ cells were seeded in 60 mm dishes for 24 h and then transiently transfected with equal amounts of DNA of each luciferase fragment using PolyJet In Vitro DNA Transfection Reagent. Twelve hours following transfection, 2.5×10⁵ cells were transferred to 24-well plates and incubated for an additional 24 h. Cells were then treated with either small-molecule Rapamycin or ABT-737 or DMSO. After 12 h of incubation, cells were lysed and 20 μL of clarified lysate was incubated with 80 μL of L-Glo luciferase assay reagent at room temperature for 20 min and subsequently read using a luminometer with 10 s integration time. All reported data was done at least in duplicate and repeated on different days. Data from luciferase assays were all normalized by lysate load and with background correction of equivalently treated mock transfected HEK293T cells.

Results

Design of Luciferase Fragmentation Sites

Referring to FIGS. 1C-1D and Table 2, identification of potential new fragmentation sites for firefly luciferase (Fluc) was achieved by a sequence dissimilarity (SD) based method that aligns similar enzymes and then identifies dissimilar sites present within less structured regions of a protein. The SD-based approach was successfully used for the identification of fragmentation sites in both human protein kinases and phosphatases. The major difference when applying the SD approach to the luciferase enzyme from firefly is that unlike human protein kinases, where the 500-member human kinome can be aligned, there is only one Fluc. Thus, the SD method was modified by a judicious choice of starting luciferase sequences available from the insect kingdom (FIG. 6). The webserver, SANSparallel, was used to search protein sequence databases, such as UniprotKB, and identify homologous sequences based on the input sequence queried. Photinus pyralis (H1AD96) was used as the input against UniprotKB database and luciferase sequences from different insects with identities varying from 88% to 48% were selected, shown in Table 3.

TABLE 3
Selection of insect luciferase from SANSparallel web server
searched against UniprotKB database for sequences homologous
to Photinus pyralis (H1AD96) for protein sequence alignment.
Luciferase was chosen whose protein sequence identity differed
from that found in Photinus pyralis to approximately 0.5.
Sequences are shown in Table 4 below.

| | Sequence | |
|---|---|---|
| Organism | Identity | UniProtKB Identifier |
| Photinus pyralis | 1.0 | H1AD96_PHOPY |
| Lucidina biplagiata | 0.88 | D5MS63_9COLE |
| Nyctophila cf. caucasica | 0.85 | Q2VL10_9COLE |
| Lampyris noctiluca | 0.84 | Q2TNU5_9COLE |
| Pyrocoelia pygidialis | 0.84 | B5A991_9COLE |
| Diaphanes pectinealis | 0.83 | Q207B1_9COLE |
| Cratomorphus distinctus | 0.83 | Q5USC8_9COLE |
| Cyphonocerus ruficollis | 0.76 | G9M6I8_9COLE |
| Drilaster axillaris | 0.76 | G9M6I9_9COLE |
| Photuris pennsylvanica | 0.70 | Q94697_PHOPE |
| Aquatica lateralis | 0.69 | H1AD94_LUCLA |
| Stenocladius azumai | 0.68 | H1AD93_9COLE |
| Hotaria unmunsana | 0.68 | Q8T6U3_9COLE |
| Lampyroidea maculate | 0.66 | Q1WLP6_9COLE |
| Phrixothrix vivianii | 0.56 | Q9U4U8_9COLE |
| Brasilocerus sp. | 0.55 | D2DHG8_9COLE |
| Rhagophthalmus ohbai | 0.54 | Q05KC0_9COLE |
| Taximastinocerus sp. | 0.54 | E3SH50_9COLE |
| Photophorus jansonii | 0.48 | R4WV23_9COLE |

TABLE 4
Sequences from insect luciferase proteins that were aligned
in Table 3. FIG. 6 features a CLustalO alignment of insect
luciferases (SEQ ID NO: 15-49 respectively, in order of
appearance listed below) with chosen split-sites (fragment
sites) of firefly Photinus pyralis luciferase (H1AD96;
SEQ ID NO: 15) bolded. Loop regions of luciferase from
Photinus pyralis luciferase (H1AD96; SEQ ID NO: 15) are
underlined.

| | | SEQ |
|---|---|---|
| Organism | Sequences | ID NO: |
| H1AD96 | MEDAKNIKKGPAPFYPLEDGTAGEQLHKAMKRYALVPGTIAFTDAHIEVNI | 15 |
| Photinus | TYAEYFEMSVRLAEAMKRYGLNTNHRIVVCSENSLQFFMPVLGALFIGVA | |
| pyralis | VAPANDIYNERELLNSMNISQPTVVFVSKKGLQKILNVQKKLPIIQKIIIMDSK | |
| | TDYQGFQSMYTFVTSHLPPGFNEYDFVPESFDRDKTIALIMNSSGSTGLP | |
| | KGVALPHRTACVRFSHARDPIFGNQIIPDTAILSVVPFHHGFGMFTTLGYLI | |
| | CGFRVVLMYRFEEELFLRSLQDYKIQSALLVPTLFSFFAKSTLIDKYDLSNL | |
| | HEIASGGAPLSKEVGEAVAKRFHLPGIRQGYGLTETTSAILITPEGDDKPG | |
| | AVGKVVPFFEAKVVDLDTGKTLGVNQRGELCVRGPMIMSGYVNNPEATN | |
| | ALIDKDGWLHSGDIAYWDEDEHFFIVDRLKSLIKYKGYQVAPAELESILLQH | |
| | PNIFDAGVAGLPDDDAGELPAAVVVLEHGKTMTEKEIVDYVASQVTTAKK | |
| | LRGGVVFVDEVPKGLTGKLDARKIREILIKAKKGGKSKL | |
| G8GE17 | MEKEKQNAGIIYGEQNFSFPNHMNYGKFLLERLSKEKAKVALINGASGEK | 16 |
| Bombyx mori | LTFGEMTQQIVNIASAIKQLGIGKGDIVAICSENRTEYLTTTIAVLCTGATVT | |
| | FINSAYSKNEFIHTTKISKPKYIFLSPSAYKEFYVTLKKLNIQKFFLYGISDD | |
| | KDVISFRDLATKYTHFNDFRPKDFNGTDSTSIILYSSGTTGLPKGVKLTHR | |
| | NLLVSGLQKPFCQKDLVILTLAPWCNTVGIILTLSCLSKNRTVLYLNKFQED | |
| | VYLQCIEKYKAGILLMVPPLLVILTKSKLIDKYDVSSVQIMYCGGAPLDSSV | |
| | MTSIKKRFTGLKHVLQGYGMTETTGALTEEREHSNKLGSVGKVVEGNIVK | |
| | VVDVETRKTLGPNQNGEICVKGPVLFEDYIGKDINEDLDEDGFYKTGDIAY | |
| | YDNEGYFYIVDRIKELIKYKAGQVAPSELEAILLQHDAVQDVGVAGAPDPL | |
| | VGELPTAFVVKKPNSKVTEKELIDFVAARVSSWKQLRGGVRFVNEIPKTG | |
| | SGKILRRILRDSLTKPPASKL | |
| A0A194R162 | MLRNSKYLFSPDEHFVPSKLHLGDFFIKTFKKYGDKVALINGATDTAVTYN | 17 |
| Papilio | DLLQASMNISVSLSQLGLKKGETVGICSESRLETWSVIVGAVCAGGVMTP | |
| machaon | LNIRYVKDELRHVMNISKPKFLFCSKQAYDNHEAIFKSLVYVENIILFGDER | |
| | VKGTLLYKDLAMISPNSMLKENVNFEEFEVVDVQGQIDTVFLMYSSGTTG | |
| | LPKGVMQTHLNALIVFNLSCLLDPTQIVLNVTSWYHAMGFMSTFIYLSHGC | |
| | KHIFLPKFETDLYLKTIEKYKVNLLVLVPPVLMTLIKTSSQYDLSSVVAIVSA | |
| | AAALRKETIAAVNEKFKNLKGICQGYGMTETTFVVTMDTLFTDGTCKPGS | |
| | VGKVVPNTLIKIADVETREPLGPYQNGEVCIKSPLILKDYIGHDKNCCLDKD | |
| | GFFRTGDVGYYDEDKHLFIVDRIKELIKYKAYQVPPAEIEAVLLQHEGVRD | |
| | VGVVGLPHPEAGELPVAFVVPQAGANPTEAELQHFVAERLSNPKHLRGG | |
| | VHFVKEIPKTASGKILRKELRELAINF | |
| A0A212FIH9 | MLRNSNYVYGDRSLSVPADLNFGSYIIDAIWKHGDKTAMTNCATNQCLSY | 18 |
| Danaus | RAMAVEAMNLSSSLTARGVKRGDVIAISSENRMEYFPTLLGICCTGATVTT | |
| plexippus | TNPTYTKDEHKYVLDISKPKLVFCSPSAYKAHGKLFHSLPYIKSVVVYGERI | |
| plexippus | PNTLSYEDLIRDNGVITPENFMAKDVEGQNDAAIVLYSSGTTGLPKGVMLT | |
| | HLNVIVTCLVAPSVDPNELTLYITPWYHSMGUASLRVFSKGNVMLFLPKFE | |
| | CDQYLRTIENYKVGQLVVAPPVLVALSKYSAKYDVSSVVAAFSGAAPLKK | |
| | ETIDAAKKRFPNLKHVFQGYGMTETTFSVIRDTYDSAHLSKTGGAGTIAAC | |
| | VVIKIVDIETRRPLGPNCRGEICVKGAPIMRGYVGRDRGDDFDDEGFFKTG | |
| | DIGYYDDDKYFFVVDRLKELIKYKGYQVPPAEIEAVLTQHPDILEAGVVGV | |
| | PHEGGEAPLAFVARRPGSNLTVEEVKSYVAEKLSNPKRLRGGVRFVEDIP | |
| | KNPSGKILRKKLRGMLKNKSKL | |
| R4WV23 | MKREKNVVYGPEPLQPLENITAGEMLYKALKRHSHLPQALVDVIGQESLS | 19 |
| Photophorus | YKEFFDLTCQLGQSLHNCGYKLSDVVSICAENNKRFFVPIIAAWYVGMIVA | |
| jansonii | PVNEDYIPDELCKVMGISKPILVFTTRKILPKVLEVQKRLSHIKRIIILDSPENL | |
| | HGCECLSNFIARFSDGHTTFFKPMHYDPAEQVAAILCSSGTTGLPKGVMQ | |
| | THRNICVRLLHASDPRSGTQLIPGVSVLAYLPFFHAFGFSINLGYFMVGLRI | |
| | IMLRRFDQETFLKAIQDYEVRSVINVPAIILFLSKSPLVDKYDLSSLKELCCG | |
| | AAPLAREIGELAAKRLNLPGVRCGFGLTESTSANIHSLPDYIKPGSLGTVTP | |
| | LMKVKILNRDTGEALGPNQVGELCVKGPMVSKGYVNNIEATKEAIDEDGW | |
| | LHSGDFGYYDEEEHFYVVDRYKELIKYKGYQVAPAELEEILLKHPCIRDAA | |
| | VVGIPDVAAGELPSAFVVKQPGKEVTEKEVYDFLAERVSHSKYLRGGVRF | |
| | VDEIPRNVTGKITRKELLKQLLNKTSKL | |
| Q718E3 | MMKREKNVIYGPEPLHPLEDKTAGEMLFRALRKHSHLPQAIVDVFGDESL | 20 |
| Pyrophorus | SYKEFFEATCLLAQSLHNCGYKMNDVVSICAENNKRFFIPIIAAWYIGMIVA | |
| plagiophthalamus | PVNESYIPDELCNVMGISKPQIVFCTKNILNKVLEVQSRTNFIKRIIILDTVEN | |
| | IHGCESLPNFISRYSDGNIANFKPLHYDPVEQVAAILCSSGTTGLPKGVML | |
| | THQNICVRLIHALDPEAGTQLIPGVTVLVYVPFFHAFGFGINLGYFMVGLRV | |
| | IMLRRFEQEAFLKAIQDYEVRSIVNVPAIILFLSKSPLVDKYDLSSLRELCCG | |
| | AAPLAKEVAEIAVKRLNLPGIRCGFGLTESTSANIHSLGDEFKSGSLGRVT | |
| | PLMAAKIADRETGKALGPNQVGELCIKGPMVSKGYVNNVKATKEAIDDDG | |
| | WLHSGDFGYYDEDEHFYVVDRYKELIKYKGSQVAPAELEEILLKNPCIRDV | |
| | AVVGIPDLEAGELPSAFVVIQPGKEITAKEVYDYLAERVSHTKYLRGGVRF | |
| | VDSIPRNVTGKITRKELLKQLLEKSSKL | |
| D2KV87 | MDKEKYILHGPESIWYVPKTTAGQQVYNALVRHSHLPEAMIDAHIQQKVS | 21 |
| Pyrophorus | YKELLETTCRLAQSLQRCGYKQNDVISICSENNLNFHCPAIAALYLGIITAPL | |
| angustus | EGYIEGELHNALNLSKPKLIFCSTKLLPKMQAMKQKFAFIKKLIILDVDEDIG | |
| luscus | SNESLSNFILRNSDASYKNFRPLDFDSNEQVAFILCSSGTTGLPKGVMLTH | |
| | TNIAVRFAHARDPRIGTQTIPGTTVLSFMPFFHALGFITTLEYFLLGLRVIML | |
| | KKFDSELFLKSIQDYEVRSMIIVPLIVSFLAKSPLVDKYDLSSLKQISCGAAP | |
| | LGKEVGDALLKRLNLEGISQGYGLTELTVAVTLTPDNEFRPGSSGAVVPF | |
| | MSAKVIDNDTGKPLGPGVTGELYFKGGLVMKGYVGNISATKEMIDENGW | |
| | LRTGDLGYYDKEGHFYIKGRLKELIKYKGFPVPPAELEALLLTHPCIKEAAV | |
| | IGIPDKSAGELPAAFVVKQPGKQITEKEVYDFVAGQISSPKHLRGGVRFID | |
| | EIPKNATNKIKRDVLRDLVTKMKSKL | |
| A9ZPM4 | MSKESNIVYGPVGAAPVLESTAGKQLFDSLKRHGHLPQAIIDYQTKOSISY | 22 |
| Agrypnus | KNLFEATCKLAHSLEEYGLKQNDVIAICSENNLNFYKPVCAALYCGIVIAPL | |
| binodulus | NDSYSEGEYVNALNISEPKLIFCSKKCLPRLVGLKARCSFIKGFVVIDSTEDI | |
| binodulus | NGNECLPNFILRNSDPNFDIEKYEPRVFNSNEQVAAILLSSGTTGFPKGVM | |
| | LTHKNFSILFAHANDPVSGTQRIPGTTVLSILPYFHGFGFITNISYIKSGIRVV | |
| | MLQRFEPEAFLRAIEEYEVRSTITVPPILIFLAKSPIVDKYNLSSLKEIICGAA | |
| | PSGREIVEAVVKRLKVSGIRYGYGLTECGLAICTTPPNNFKIGSSGVVVPF | |
| | MAVKIRDVESGKTLKPTQIGEICVKGDMLMKGYAGNEKATKEMIDEDGWL | |
| | HTGDIGYFDKDGHIYIDRIKELIKYKGFQVPPAELEALLLHHPCVKDAAVIG | |
| | IPDELAGELPAAFIVKQHGKEVTEKEIDYIAKQVSSAKHLRGGVRFIPDIP | |
| | RTAAGKIQRNLLRNMIAKKKSKL | |
| Q05KC0 | MPNEIILHGAKPRDPLDLGTAGIQLYRALTNFSFLREALIDAHTEEVVSYADI | 23 |
| Rhagophthalmus | LENSCRLAKCYENYGLRQNSVISVCSENSTIFFYPVIAALYMGVITATVNDS | |
| ohbai | YTERELLETLNISKPELVFCSKKAIKNMMALKRNVNFIKKVVLLDSKEDMG | |
| | EAQCLSNFMARYSEPNLDVRNFKPRDFDAKEQVALIMSSSGTTGLPKGV | |
| | VLTHRNLSVRFVHCKDPLFGTRTIPSTSILSIVPFHHAFGMFTTLSYFIVGLR | |
| | VLLKRFEEKFFLSTIEKYRIPTIVLAPPVMVFLAKSPLVDQYDLSSIREVAT | |
| | GGAPVGTEVAVAVAKRLKIGGILQGYGLTETCCAVLITPHDDVKTGSTGRV | |
| | APYVQAKIVDLTTGKSLGPNKRGELCFKSEIIMKGYFNNKQATEEAIDKEG | |
| | WLHSGDVGYYDDDGHFFVVDRLKELIKYKGYQVAPAELEWLLLQHPSIKD | |
| | AGVTGVPDEAAGELPGACIVLQEGKSLTEQEIDYIAERVSPTKRIRGGVVF | |
| | VDDIPKGATGKLVRSELRKLLAQKKSKL | |
| Q9U4U8 | MEEENIRHGERPRDIVHPGSAGQQLYQSLYKFASFPEAIIDAHTNEVISYA | 24 |
| Phrixothrix | QIFETSCRLAVSIEQYGLNENNVVGVCSENNINFFNPVLAALYLGIPVATSN | |
| vivianii | DMYTDGELTGHLNISKPTIMFSSKKALPLILRVQQNLSFIKKVVVIDSMYDIN | |
| | GVECVSTFVARYTDHTFDPLSFTPKDFDPLEKIALIMSSSGTTGLPKGVL | |
| | SHRSLTIRFVHSRDPIYGTRTVPQTSILSLVPFHHAFGMFTTLSYFVVGLKV | |
| | VMLKKFEGALFLKTIQNYKIPTIVAPPVMVFLAKSPLVDQYDLSSLTEVAT | |
| | GGAPLGKDVAEAVAKRLKLPGIIQGYGLTETCCAVMITPHNAVKTGSTGR | |
| | PLPYIKAKVLDNATGKALGPGERGEICFQSEMIMKGYYNNPEATIDTIDKD | |
| | GWLHSGDIGYYDEDGNFFIVDRLKELIKYKGYQVAPAELENLLLQHPSIAD | |
| | AGVTGVPDEFGGQLPAACVVLESGKTLTEKEVQDFIAAQVTPTKHLRGGV | |
| | VFVDSIPKGPTGKLIRKELREIFAQRAPKSKL | |
| D2DHG8 | MEEENIRYGDRPRDIVHPGSAGEQLYQSLYKFSYFPEAIVDAHTNEKISYA | 25 |
| Brasilocerus | TIFETSCRLAVSIEKLGLNERNVVGVCSESNLNFFNPVLAALYLGIPVATSN | |
| sp. | DMYTDGELSGHLNISKPDIMFCSKKALPLILRAQKNLNFIKKVVVIDSMFNI | |
| | DGVECVHSFVSHYTDSSFDPLSFKPKQFDPLQEIALIMSSSGTTGLPKGV | |
| | VLSHRSLTIRFVHSRDPLYGTRTIPETSSLSLVPFHHAFGMFTTLSYFWGL | |
| | RIVMLKKFEGDLFLKTIQNYKIPTIVVAPPVMVFLAKSPLVDKYDLSSLREV | |
| | ATGGAPLGKDVGEAVAKRLGLSGVLQGYGLTETCCAVVITPHNNVKTGS | |
| | AGKVVPYVSAKVLDKATGKALGPKERGEICFKSEMLMKGYYNNPEATRE | |
| | TIDEDGWLHSGDIGYYEEDGMFYIVDRLKELIKYKGYQVAPAELENLLLQH | |
| | PDIADAGVTGIPDEFAGQLPAACVVLEPGKTLTEKEAMDFIAEQVTPTKHL | |
| | RGGVIFVDRIPKGPTGKLIRNELRAIFAKKAASSKL | |
| E3SH50 | MEEENIMHGAEPRDLVHPGSAGTQLYRSLYKFSYFPEAIVDAHTHEVISYA | 26 |
| Taximastinocerus | TIFNVSCRLAASLEKYGLGKEDDVVGVCSENNLNFFSPVLAALYLGVPVAT | |
| sp. | SNDMYTDGEISGHLNISKPCIMFCSKKALPLILKVQKNLHFMKKVVVIDSMY | |
| | DMNGVECINTFMNHYTDHTFDPLKFQPKEFDPLKKTALIMSSSGTTGLPK | |
| | GVLSHRSLTIRFVHSRDPIYGTRTIPETSILSLVPFHHAFGMFTTLSYFVV | |
| | GLRIVMLKKFEGELFLKTIQHYKIPTIVVAPPVMVFLAKSPLVDKYDLSSLRE | |
| | VATGGAPIGKDVAHAVAKRLGLSGILQGYGLTETCCAVVITPHDNLRTGSA | |
| | GKVVPYVKAKILDKATGKALGPNERGEICFKSEMLMKGYHNNPEATRETI | |
| | DEDGWLHSGDIGYYEEDGTIYIVDRLKELIKYKGYQVAPAELENLLLQHPDI | |
| | ADAGVTGVPDGFAGQLPAACIVLEPGKTLTEKEVVDFLAERVTPTKYLRG | |
| | GVIFVDRIPKGPTGKLMRQELRAIFAAKKAQTSKL | |
| H1AD94 | MENMENDENIVYGPEPFYPIEEGSAGAQLRKYMDRYAKLGAIAFTNALTG | 27 |
| Aquatica | VDYTYAEYLEKSCCLGEALKNYGLVVDGRIALCSENCEEFFIPVLAGLFIGV | |
| lateralis | GVAPTNEIYTLRELVHSLGISKPTIVFSSKKGLDKVITVQKTVTAIKTIVILDS | |
| | KVDYRGYQSMDNFIKKNTPPGFKGSSFKTVEVNRKEQVALIMNSSGSTGL | |
| | PKGVQLTHENAVTRFSHARDPIYGNQVSPGTAILTVVPFHHGFGMFTTLG | |
| | YLTCGFRIVMLTKFDEETFLKTLQDYKCSSVILVPTLFAILNRSELLDKYDLS | |
| | NLVEIASGGAPLSKEIGEAVARRFNLPGVRQGYGLTETTSAIIITPEGDDKP | |
| | GASGKVVPLFKAKVIDLDTKKTLGPNRRGEVCVKGPMLMKGYVDNPEAT | |
| | REIIDEEGWLHTGDIGYYDEEKHFFIVDRLKSLIKYKGYQVPPAELESVLLQ | |
| | HPNIFDAGVAGVPDPIAGELPGAVVVLEKGKSMTEKEVMDYVASQVSNAK | |
| | RLRGGVRFVDEVPKGLTGKIDGKAIREILKKPVAKM | |
| E3WI50 | MENMENDENIVVGPKPFYPIEEGSAGTQLRKYMERYAKLGAIAFTNAITGV | 28 |
| Luciola | DYSYAEYLEKSCCLGKALQNYGLVVDGRIALCSENCEEFFIPVIAGLFIGVG | |
| cruciate | VAPTNEIYTLRELVHSLGISKPTIVFSSKKGLDKVITVQKTVTTIKTIVILDSKV | |
| | DYRGYQCLDTFIKRNTPPGFQASSFKTVEVDRKEQVALIMNSSGSTGLPK | |
| | GVQLTHENTVTRFSHARDPIYGNQVSPGTAVLTVVPFHHGFGMFTTLGYL | |
| | ICGFRVVMLTKFDEETFLKTLQDYKCTSVILVPTLFAILNKSELLNKYDLSNL | |
| | VEIASGGAPLSKEVGEAVARRFNLPGVRQGYGLTETTSAIIITPEGDDKPG | |
| | ASGKVVPLFKAKVIDLDTKKSLGPNRRGEVCVKGPMLMKGYVNNPEATK | |
| | ELIDEEGWLHTGDIGYYDEEKHFFIVDRLKSLIKYKGYQVPPAELESVLLQH | |
| | PSIFDAGVAGVPDPVAGELPGAVVVLESGKNMTEKEVMDYVASQVSNAK | |
| | RLRGGVRFVDEVPKGLTGKIDGRAIREILKKPVAKM | |
| Q8T6U3 | MEMEKEENVVYGPLPFYPIEEGSAGIQLHKYMQQYAKLGAIAFSNALTGV | 29 |
| Hotaria | DISYQEYFDITCRLAEAMKNYGMKQEGTIALCSENCEEFFMPVLAGLYIGV | |
| unmunsana | AVAPTNEIYTLRELNHSLGIAQPTIVFSSRKGLPKVLEVQKTVTCIKTIVILDS | |
| | KVNFGGYDCMETFIKKHVELGFRPTSFVPIDVKNRKQHVALLMNSSGSTG | |
| | LPKGVLITHEGTVTRFSHAKDPIYGNQVSPGTAILTVVPFHHGFGMFTTLG | |
| | YFACGYRVVMLTKFDEELFLRTMQDYKCTSVILVPTLFAILNKSELIDKFDL | |
| | SNLTEIASGGAPLAKEVGEAVARRFNLPGVRQGYGLTETTSAFIITPEGDD | |
| | KPGASGKVVPLFKVKVIDLDTKKTLGVNRRGEICVKGPSLMLGYLNNPEA | |
| | TKETIDDEGWLHTGDIGYYDEDEHFFIVDRLKSLIKYKGYQVPPAELESVLL | |
| | QHPNIFDAGVAGVPDSEAGELPGAVVVMEKGKTMTEKEIVDYVNSQVVN | |
| | HKRLRGGVRFVDEVPKGLTGKIDAKVIREILKKPQAKM | |
| Q1WLP6 | MEMEKEENVVYGPQPFYPIEKGSAGIQLHKYMHQYAQLGAIAFSNALTGV | 30 |
| Lampyroidea | DISYQEYFDISCRLAEAMENYGMKPEGRIALCSENCEEFFIPVLAGLYIGV | |
| maculate | GVAPTNEIYTLRELNHTLGIAEPTIVFSSKKGLPKVLEVQKTVTCIKTIVILNS | |
| | KVNFGGYDCVETFIKKNVELGFQPTSFKPIDVKNRKEHVALIMNSSGSTGL | |
| | PKGVQITHEATVTRFSHAKDPIYGNQVSPGTAILTVVPFHHGFGMFTTLGY | |
| | FACGYRIVMLTKFDEEIFLKTMQDYKCTSVSLVPTLFGILNKSELIDKFDLSN | |
| | LTEIASGGAPLAKEVGEAVARRFNLPGVRQGYGLTETTSAFIITPEGDDKP | |
| | GASGKVVPLFKVKVIDLDTKKTLGANRRGEICVKGPSLMKGYINNPEATKE | |
| | IIDEEGWMHTGDIGYYDEDEHFFIVDRLKSLIKYKGYQVPPAELESVLLQH | |
| | PDIFDAGVAGVPDPEAGELPGAVVVMEKGKTMTEKEIVDYVNSQVVNHK | |
| | RLRGGVRFVDEVPKGLTGKIDAKVIREILKKPQAKM | |
| Q94697 | MSIENNILIGPPPYYPLEEGTAGEQLHRAISRYAAVPGTLAYTDVHTELEVT | 31 |
| Photuris | YKEFLDVTCRLAEAMKNYGLGLQHTISVCSENCVQFFMPICAALYVGVAT | |
| Pennsylvanica | APTNDIYNERELYNSLSISQPTVVFTSRNSLQKILGVQSRLPIIKKIIILDGKK | |
| | DYLGYQSMQSFMKEHVPANFNVSAFKPLSFDLDRVACIMNSSGSTGLPK | |
| | GVPISHRNTIYRFSHCRDPVFGNQIIPDTTILCAVPFHHAFGTFTNLGYLIC | |
| | GFHVVLMYRFNEHLFLQTLQDYKCQSALLVPTVLAFLAKNPLVDKYDLSN | |
| | LHEIASGGAPLSKEISEIAAKRFKLPGIRQGYGLTETTCAIVITAEGEFKLGA | |
| | VGKVVPFYSLKVLDLNTGKKLGPNERGEICFKGPMIMKGYINNPEATRELL | |
| | DEEGWIHSGDIGYFDEDGHVYIVDRLKSLIKYKGYQVPPAELEALLLQHPFI | |
| | EDAGVAGVPDEVAGDLPGAVVVLKEGKSITEKEIQDYVAGQVTSSKKLRG | |
| | GVEFVKEVPKGFTGKIDTRKIKEILIKAQKGKSKSKAKL | |
| D5MS63 | MEEDKNILRGPAAFYPLEDGTAGEQLHRAMKRYALIPGTIAFTDAHAGVNI | 32 |
| Lucidina | TYSEYFEMACRLAESLKRYGLGLQHRIVVCSENSLQFFMPVVGALFIGVG | |
| biplagiata | VAPANDIYNERELLNSMTISQPTLVFCSRKGLQKILNVQKKLPVIQKIIILDTK | |
| | EDYMGFQSMYSFVDSQLPVGFNEYDYVPDSFDRDQATALIMNSSGSTGL | |
| | PKGVELTHTSVCVRFSHCRDPVFGNQIIPDTAILSVIPFHHGFGKFTTLGYL | |
| | ICGFRVVLMYRFEEELFLRSLQDYKIQSALLVPTLFSFFAKSTLIDKYDLSNL | |
| | HEIASGGAPLAKEVGEAVAKRFNLRGIRQGYGLTETTSAVIITPEGDDKPG | |
| | AVGKVVPFFSAKVVDLDTGKTLGVNQRGELCLKGPMIMKGYVNNPEATN | |
| | ALIDKDGWLHSGDISYWDEDGHFFIVDRLKSLIKYKGYQVPPAELESILLQ | |
| | HPFIFDAGVAGIPDDEAGELPAAVVVLEEGKTMTEKEIMDYVAGQVTTAK | |
| | RLRGGVVFVDEVPKGLTGKLDARKIREILVKAKKTKSKL | |
| Q5USC8 | MEEDKNIMYGPAPFSPLEEGTAGEQLHKAMKRYAQIPGTIAFTAAHVEVN | 33 |
| Cratomorphus | VTYAEYFEMACRLAETMKRYGLGLDHRIAVCSENSLQFFMPVCGALFIGV | |
| distinctus | GVAPTNDIYNERELYNSLSISQPTVVFCSKRALQKILGVQKSLPVIKKIVILD | |
| | SREDYMGKQSMYSFIQSYLPGGFNEYDYVPDTFDRDMATALIMNSSGST | |
| | GLPKGVELSHKNVCVRFSHCRDPVFGNQIPDTAILTVIPFHHGFGMFTTL | |
| | GYLTCGFRIVLMYRFEEELFLRSLQDYKIQSALLVPTLFSFFAKSTLVDKYD | |
| | LSNLHEIASGGAPLAKEVGEAVAKRFKLPGIRQGYGLTETTSAIIITPEGDD | |
| | KPGACGKVVPFFAAKIVDLDTGKTLGVNQRGELYVKGPMIMKGYVNNPE | |
| | ATNALIDKDGWLRSGDIAYYDEDGHVFIVDRLKSLIKYKGYQVPPAELESIL | |
| | LQHPFIFDAGVAGIPDEDAGELPAAVVVLEEGKTMTEQEVMDYVAGQVTA | |
| | SKRLRGGVKFVDEVPKGLTGKIDSRKIREILVMGKKSKL | |
| Q207B1 | MEDDKNIIYGPPPFYPLEDGTAGEQLHRAMKRYAQVPGTIAFTDAHAEVNI | 34 |
| Diaphanes | TYSEYFELSCRLAEAMKRYGLGLQHRIAVCSENSLQFFIPVCSALYIGAGV | |
| pectinealis | APTNDIYNERELYNSLSISQPTIVFCSKRALQKILGVQKKLPVIEKIVILDSRE | |
| | DYMGKQSMYSFIDSHLPAGFNEYDYKPDSFDRDTATALIMNSSGSTGLPK | |
| | GVDLSHKNVCVRFSHCRDPVFGNQIIPDTAILTVIPFHHGFGMFTTLGYLT | |
| | CGFRVVLMYRFEEELFLRSLQDYKIQSALLVPTLFSFFAKSTLVDKYDLSN | |
| | LHEIASGGAPLAKEVGEAVAKRFKLPGIRQGYGLTETTSAIIITPEGDDKPG | |
| | ACGKVVPFFCGKIVDLDTGKTLGVNQRGELCVKGPMIMKGYINNPEATNA | |
| | LIDKDGWLHSGDIAYYDKDGHFFIVDRLKSLIKYKGYQVPPAELESILLQHP | |
| | FIFDAGAAGIPDPDAGELPAAVVVLEEGKTMTEQEVMDYVAGQVTASKRL | |
| | RGGVKFVDEVPKGLTGKIDSRKIREILMMGKKSKL | |
| B5A991 | MEDDHKNIMHGPAPFYPLEDGTAGEQLHKAMKRYAQVPGTIAFTDAHVE | 35 |
| Pyrocoelia | VNITYSEYFEMACRLAETMKRYGLGLQHHIAVCSENSLQFFMPVCGALFI | |
| pygidialis | GVGVAPTNDIYNERELYNSLSISQPTIVFCSKRALQKILGVQKKLPVIEKIVIL | |
| | DSREDYMGKQSMYSFIESHLPAGFNEYDYIPDSFDRDTATALIMNSSGST | |
| | GLPKGVELTHKNVCVRFSHCRDPVFGNQIIPDTAILTVIPFHHGFGMFTTL | |
| | GYLTCGFRIVLMYRFEEELFLRSLQDYKIQSALLVPTLFSFFAKSTLVDKYD | |
| | LSNLHEIASGGAPLAKEVGEAVAKRFKLPGIRQGYGLTETTSAIIITPEGDD | |
| | KPGACGKVVPFFSAKIVDLDTSKTLGVNQRGELCVKGPMIMKGYVNNPEA | |
| | TSALIDKDGWLHSGDIAYYDKDGHFFIVDRLKSLIKYKGYQVPPAELESILL | |
| | QHPFIFDAGVAGIPDPDAGELPAAVVVLEEGKTMTEQEVMDYVAGQVTAS | |
| | KRLRGGVKFVDEVPKGLTGKIDSRKIREILTMGKKSKL | |
| Q2VL10 | MEDAKNIMHGPPPFYPLEDGTAGEQLHKAMKRYAQVPGTIAFTDAHAEV | 36 |
| Nyctophila cf. | NITYSEYFEMACRLAETMKRYGLGLQHHIAVCSENSLQFFMPVCGALFIG | |
| caucasica | VGVAPTNDIYNERELYNSLSISQPTIVFCSKRALQKILGVOKKLPIIQKIVILD | |
| | SREDYMGKQSMYSFIESHLPAGFNEYDYIPDSFDRETATALIMNSSGSTG | |
| | LPKGVELTHKNICVRFSHCRDPVFGNQIIPDTAILTVIPFHHGFGMFTTLGY | |
| | LTCGFRIVLMYRFEEELFLRSLQDYKIQSALLVPTLFSFFAKSTLVDKYDLS | |
| | NLHEIASGGAPLAKEVGEAVAKRFKLPGIRQGYGLTETTSAIIITPEGDDKP | |
| | GACGKVVPFFSAKIVDLDTGKTLGVNQRGELCVKGPMIMKGYVNNPEAT | |
| | SALIDKDGWLHSGDIAYYDKDGHFFIVDRLKSLIKYKGYQVPPAELESILLQ | |
| | HPFIFDAGVAGIPDPDAGELPAAVVVLEEGKTMTEQEVMDYVAGQVTASK | |
| | RLRGGVKFVDEVPKGLTGKIDARKIREILMMGKKSKL | |
| Q2TNU5 | MEDAKNIMHGPAPFYPLEDGTAGEQLHKAMKRYAQVPGTIAFTDAHAEV | 37 |
| Lampyris | NITYSEYFEMACRLAETMKRYGLGLQHHIAVCSENSLQFFMPVCGALFIG | |
| noctiluca | VGVAPTNDIYNERELYNSLSISQPTIVFCSKRALQKILGVQKKLPIIQKIVILD | |
| | SREDYMGKQSMYSFIESHLPAGFNEYDYIPDSFDRETATALVMNSSGSTG | |
| | LPRGVELTHQNVCVRFSHCRDPVFGNQIIPDTAILTVIPFHHGFGMFTTLG | |
| | YLTCGFRIVLMYRFEEELFLRSLQDYKIQSALLVPTLFSFFAKSTLVDKYDL | |
| | SNLHEIASGGAPLAKEVGEAVAKRFKLPGIRQGYGLTETTSAIIITPEGDDK | |
| | PGACGKVVPFFSAKIVDLDTGKTLGVNQRGELCVKGPMIMKGYVNNPEA | |
| | TSALIDKDGWLHSGDIAYYDKDGHFFIVDRLKSLIKYKGYQVPPAELESILL | |
| | QHPFIFDAGVAGIPDPDAGELPAAVVVLEEGKTMTEQEVMDYVAGQVTAS | |
| | KRLRGGVKFVDEVPKGLTGKIDGRKIREILMMGKKSKL | |
| H1AD93 | MPSSMMSKKDLEDKNVVHGPDPYYLVDEGNAGQQLHKTILRYAQLPDTI | 38 |
| Stenocladius | AFTDGHTKRDVTYAHYFDLTCRLAESLKRYGLNLQSRIAVCSENNVEFFIP | |
| azumai | VVASLYLGVGVAPTNDIYNETELFNSLNISQPTIVFVSKRALHKILEVKKRIPI | |
| | IKTVVVLDTEEDFMGYHCLHSFMKHYLPPNFDIMSYKPEEFARDGQLALIM | |
| | NSSGSTGLPKGVMLAHRSVVRFSHCKDPVFGNQIIPDTAILTVIPFHHGF | |
| | GMFTTLGYLTCGFRIVLLRKFDEHYFLKCLQDYKIQSALLVPTLFSFFAKST | |
| | LVDQYDLSNLKEIASGGAPLAKEVGEAVAKRFKLPGIRQGYGLTETTSAVII | |
| | TPEGEDKPGSTGKVVPFFSAKIVDLNNGKSVGPHQRGELCLKGDMIMMG | |
| | YCNNKAATDEMIDKDGWLHSGDIAYYDEDGHFFIVDRLKSLVKYKGYOVA | |
| | PAELEAVLLQHPCVFDAGVTGVPDDVAGELPGACVLEKGKHVTEQEVM | |
| | DYVAGQLSSYKKLRGGVRFIDEIPKGLTGKIDRKALKEILKKPQSKM | |
| A0A1B3TNR3 | MEDKNIVHGPPPVYPVDDGTAGQQLHKTILRYAKLPGTIAFTNAHTEVNVT | 39 |
| Phausis | YSEYFDMTCRLAEAMKRFGLDLKHNIAVCSENSLEFFVPVVAALYLGVGV | |
| reticulata | APTNDIYNERELFNSLNISQPTIVFSSKRAVHKIMEVRKQLPVIKKIIILDTKD | |
| | DFMGMHSMHSFIKAHIPSSFNEMDFKPESFDRSDVVALIMNSSGSTGLPK | |
| | GVALSHKNVVVRFSHCKDPVFGNQIVPDTAILTVIPFHHGFGMFTTLGYLT | |
| | CGFRIVLMNKFEEELFLRSLQDYKIQSALLVPTLFSFFAKSTLVDKYDLSHL | |
| | EEIASGGAPLAKEVGEAVAKRFKLPGVRQGYGLTETTSACIITPEGDDKPG | |
| | STGKVPFFSAKIIDLDTRKSLGPNQRGELCLKGDMIMKGYVNNPEATNAL | |
| | IDKDGWLHSGDIAYYDEDGHFFIVDRLKSLIKYKGYQVAPAELEAVLLQHP | |
| | FIFDAGVTGIPDEVAGQLPAACVVVEKGKTLTEKEIMDYVAGQVSSSKRLR | |
| | GGVRFVDEIPKGLTGKIDGRQLKEIFKKPQAKL | |
| G9M6I8 | MEMEDKNVRGPAPFYPVDDGTAGEQLHKTILRYAQLSGTIAFTDAHTEV | 40 |
| Cyphonocerus | NVTYAEYYDATCRLAEAMKRYGLDLKHRIAVCSENSLEFFVPVIAALYLGV | |
| ruficollis | AVAPTNDIYNERELFNSLNISQPTIVFTSKRALHKILEVQKQLPVIKKIVVLDS | |
| | KDDFMGMQSMHSFIKHYLPADFNELDYKPESFDRNEQVALIMNSSGSTG | |
| | LPKGVQLTHKNVVVRFSHCKDPVFGNQIIPDTAILTVIPFHHGFGMFTTLG | |
| | YVTCGFRIVMHKFEEELFLRSLQDYKIQSALLVPTLFSFFAKSTLVDKYDL | |
| | SNLHEIASGGAPLAKEVGEAVAKRFKLPGVRQGYGLTETTSAVIITPEGDD | |
| | KPGSTGKIVPFFSAKILDLDTKKSLGPNQRGELCLKGDMIMKGYVNNPEAT | |
| | NALIDKDGWLHSGDIAYYDEDGHFFIVDRLKSLIKYKGYQVPPAELESVLL | |
| | QHPYIFDAGVAGVPDEDAGELPGACVVLEKGKHLTAKEVMDYVAGQVSS | |
| | YKRLRGGVRFIDEIPKGLTGKIDGKALREILKKPQAKL | |
| G9M6I9 | MNMEDKNVVHGPASFYPVDDGTAGQQLHKTILRYAQLPGTIAFTDAHTE | 41 |
| Drilaster | QNLTYAEYFDLTCRLAESMKRYGLGLKHAIAVCSENSLHFFAPVIAALYLG | |
| axillaris | VAVAPTNDIYNERELYNSLNISQPTIVFCSKRALLKIMEVQKKLPVIQKIIVLD | |
| | HKEDFQGLHSMHSFIKHYLPSHFNEHDFKPEKFDRNDQVALIMNSSGSTG | |
| | LPKGVMLTHKNVVVRFSHCKDPVFGNQIIPDTAILTVIPFHHGFGMFTTLG | |
| | YLTCGFRIVVMHKFEEELFLRSLQDYKIQSALLVPTLFSFFAKSTLVDKYDL | |
| | SNLKEIASGGAPLAKEVGEAVAKRFKLPGVRQGYGLTETTSAVIITPEGED | |
| | KAGSTGKVVPFFSAKIIDLDTRQSLGPHQKGELCLKGDMIMKGYANNPEA | |
| | TNALIDKDGWLHSGDIAYYDEDGHFFIVDRLKSLIKYKGYQVAPAELESVLL | |
| | QHPYIFDAGVTGIPDEVAGELPAACVVLEKGKHLTEKEVMDYVASQVSSS | |
| | KKLRGGVRFIDEIPKGLTGKIDSRGLKEILKRPQSKM | |
| W5JL40 | MNPTHPLPTDEKFIISGPPEPANITESCSSLGMFLYKRLLRNGDTTALIDGV | 42 |
| Anopheies | YSTELQYLELLEKAIRLAESLRRHANLQAGDVVGISENRLEFPVALYAAFF | |
| darlingi | LGAAVAPINLTYTERELNHALNLSKPKVLFVSPYSAERVKVAGENRNFIE | |
| | RVFLFGDENAFGSNVTLYEQFLATTSTINPHTFPILPTNVDEHVALILCSSG | |
| | TTGLPKGVQLTQRNVMASVSLLSELEASFEVPVVVLGVIPWFHAFGCLTLL | |
| | NVLSNKLKLVSLPKFEEGLFLSCIENYRCSFVFVVPPLMVFLAKHPLVDSY | |
| | DLSCVNTLLCGAAPLSRETEELVKRRIGVKHVLQGYGMSETTLATLVQTA | |
| | ECNKPGSVGKLQVGTRAKVIDLETGKPLGPNRPGELCFQGTQIMKGYIGN | |
| | DKATRETIDQNGWLHTGDIGYYDEDYEFFIIDRLKELIKYKGYQVPPAEIESI | |
| | LLTNVKIKDAGVVGLPDELAGELPLAFVVKQPNVTLTEEEVRQYVAERTSP | |
| | AKRLHGGVRFVEEIPKNVSGKILRRELRALLKKPHAKL | |
| B0W9Q1 | MASKDESRFIISGGPEPQNITEGFGSMGAFVRNRMRRNGNDVAVIDGVY | 43 |
| Culex | GTEVQYLELLEQSVRLAECLRTLAGIRVGDVIGIVSENRLEFPAVLFGALFV | |
| quinquefasciatus | GATVAPINLTYSERELEHAFSLSKPKLIFVSPFSADRVVAAARRNRHIVQKV | |
| | VLFGDENPFVEGQDQRDVVLFEEFQRPVTFVNPLNFYIPTVDIDQHVALIM | |
| | CSSGTTGLPKGVQLTHANLLASIALLEESSNLMEPPPGGIVLLGVLPWFHA | |
| | YGCMTLINVICNKQKLVSLPKFEEGLFLSCIENYRCTMIFVVPPLVVFLAKH | |
| | PLVDSYDLSSIDTLLCGAAPLSKETEDLVKARLNVRHVRQGYGMSETTLA | |
| | TLVQNGECHKSGSVGKVQIGTLAKVIDPETGKLLGPNQHGELCFKGSQIM | |
| | KGYIGNEKATRETIDQDGWLHTGDVGYYDEDFEFFIVDRLKELIKYKGFOV | |
| | PPAEIEAILLTNPKVKDAAVIGLPDEAAGELPLAFVVKQDGVDISEAEIKKYV | |
| | ADRTSPAKRLHGGVRFIAEIPKNLSGKILRRELRAMLQTIKSKL | |
| A0A0K8VAT3 | MTTLLPGNIYAGPITCTETHAYNSLGHYILEKYKSYGTQTVMIDAVTGQE | 44 |
| Bactrocera | YTAKYMHDSVRMAHVLQHLGIKAGDVVGLSSENNVRFAITMYAAFAINA | |
| latifrons | TVAPLNITYSEREVHHAINLSKPKVIFASKLTVNVIEKVAKNNSFVKSIVVLD | |
| | PCSSNDKILNFDELMKSKKIASKDYFDTAPANKKDDVALIICSSGTTGLPKG | |
| | VQLTQYNILFTLDSNLEPTSIPMGEITLLTVIPWFHAFGCITLITTTVLGTTLV | |
| | YLPKFEENLFLGAIQKYRVLMAFMVPPLMVFLAKHPLVDKYDLSSLMVLLC | |
| | GAAPLSKDTQDLITERIGVPIIRQGYGLSESTLSLLVONDEACKPGSVGSIK | |
| | LGIYGKVIDTETGRALGPNQRGELCFKGDCIMKGYIGDTKSTQSMIIDGWL | |
| | HTGDIGYFDDDFEFFIVDRIKELIKYKGFQVPPAEIEALLLTNKKINDAAVIGK | |
| | PDEESGELPLAFVVKQPNVELSEEEVIKFVAEHASPAKRLRGGVIFVNNIP | |
| | KNLSGKILRRELRELLKNRRSKL | |
| A0A2P8Y7W7 | MDAHHILTGPEPSCPTPNTSLGRILYDSIKSHKNKVALVDAVTGETRTFSDI | 45 |
| Blattella | LSKSEKLAESILSRGVKPGDVISICSENSIDFILPVLATYYVGATCAPLNPLY | |
| germanica | TTRELIHSINISKPSIMFCSQKALKTVEEMSHKVEFLKEIIVFGQSNSHSHTS | |
| | FNSLLQNKSTNFQPIDVDPKEFIAAILCSSGTTGLPKGVMLTQHNILTVFGTI | |
| | HEPRFGSLREDDIALGLLPMFHSYAFACQLMTTYIGATVVVNGFNEEVFL | |
| | KSIQNYKITLLFLVPPLVIFLAKAKIVDNFNLSSIRSIRCGAAPLSEEVEVLLN | |
| | ERLGVGPVLQGYGMTETTLAVLVTPTDKHKTGSSGVVVPGMKCKVVDLE | |
| | TGEILGPNRKGELCFKGPLIMKGYCGNLQATSATFDKEGFLRTGDVGYYD | |
| | EDGFFFIVDRVPPAEIEALLLTHPEIKDAGVVGIPDDIAGELPLAFVVKQPDS | |
| | KVTKEEIIQYIAGQVSPQKRLHGGVEFVENIPKTASGKILRRDLKLMLKAKL | |
| A0A0L7R1M1 | MPEENILHGPPLPVMEFENLSLGQLILNQLSIHRFWVAQVNAYTGKIQTFK | 46 |
| Habropoda | DILDISRKLAIALDKEGLKKGDKIAICSENNLEFCLAVCAAFFLGVTVCPLNP | |
| laboriosa | LYTERELKHALSISKPKYIFISPFGAKNICKVAPQLFWLPKLIMLTESTDKILP | |
| | CIDSLISNTIISDNFHACSVDVNDHVAVILCSSGTTGLPKGVMLTDKNFLTVI | |
| | RNLVALLPDRLNNNSTSLALLPFFHVYSFSVMLANLIFGSNCIILPRFDEKIF | |
| | LHAIEKYKIQHLTVVPPLMVFLAKHPIVDKYNLSSIKEIWCGAASLSKEIAKM | |
| | VENRLNITIKQAYGLTETTLAVLQAPDNSTKYGSVGTLVPGISAKVIPVNGD | |
| | ESSEPLGPNSVGELCFKGDLIMKGYCNNEEATAATIDKDRWLHSGDVGY | |
| | YDEQGYFYIVDRLKELIKYKGYQVPPAELEAILLTCPGVNDAAVIGLPHEET | |
| | GELPTAFVVKQEGSDVTAEEITNFVNGKFLN | |
| E2AA43 | MSKHAHILHGKGELVAEFNFSVGQLLLRQLTTHDTRIAQIDAHTGKEQTFK | 47 |
| Camponotus | YILDTSRKLAIYLQKEGLTVNDGIAICSENNLDFCIPVCATFYLGAIVCPLNP | |
| floridanus | LYSKGELKHALTIIKPKYIFISVIALNNMISIFKELHWFPRVLLLNNYDNIDIPW | |
| | MSMDKAISNVSDDNIDKFQAVPVDINNHVTAILCSSGTTGLPKGVMWTDK | |
| | NITTIIRMYINHPFVANAMSLALLPFFHAYSFVLLVIRLIGGNSSVVFSRFEE | |
| | KLFLQSIEKYKIQYLTVVPPIMVFLAKHPLVDKYDLSSIRKIWCGAAPLSEKI | |
| | EKAVVKRLNIPEISQGYGLTETTLAVLRFPQDTALKFGSVGMLVPGVSAKV | |
| | IPLGEYETDETLGPNCEGELCFKGDLIMKGYYNDEKSTRATIDKDGWLHT | |
| | GDVGYYDEEGYFFIVDRIKELIKYKGYQVPPAELEAILLTYSGIKDAAVIGIP | |
| | NEEAGELPMAFIVKEENANIREEDIIQYVNERVSNPKRLRGGIKFVDSIPKT | |
| | PSGKILRRVLRRTLKSKL | |
| F4WWH1 | MSQQSNILRNEEELLPEFNISIGQCLLMQLTTHGTRVAHINPHTGKEQTFO | 48 |
| Acromyrmex | YILDTSRRLAVYLQREGLKVNDTIAVCSENNLEFCIPVCAAFYLGAIACPLN | |
| echinatior | PLYSEREFKHALSISKPKYIFISTVSFNGIRKIFRELHWSPKILMLTDGNNNV | |
| | SWASMYKVMSNVSIDDANALQPASVNLDDHVTAILCSSGTTGLPKGVMLT | |
| | DKNITTVIRMYMNTNTIPENAVSLSLLPFFHAYSFVFMILTILRGNCSIIFSHF | |
| | EEELFLQYIEKYKIEYMPMVPSLMVFLAKHPLVDKYDLSCVKTIWSGAAPL | |
| | SKEIQQAVAKRLNMNIIDVKQGYGLTETTLAVLRSPDGKGKLGSVGVVP | |
| | GTLVKVIPIGEYETDKALGPNCEGELCFKGDLIMKGYYNDEESTRATIDKD | |
| | GWLHTGDVGYYDEDGYFYIVDRIKELIKYKGYQVPPAELEAILLTFPGVQD | |
| | AAVIGIPNDKTGELPMAFIVKEENSNICEKDIIQYVNERVSNPKRLRGGIRF | |
| | VDSIPKTPSGKILRRVLRDRLKSKL | |
| A0A151WRK1 | MPQQSNILRGEGELLPELKISMGQLLLMQLTTHGTRVAHINPHTGKEQTF | 49 |
| Trachymyrmex | QHILDTSRRLAVYLQREGLKANDTIAVCSENNLEFCIPVCAAFYLGAIACPL | |
| zeteki | NPLYSERELKHALFISKPKYIFISMISFNGMRKVFRELHWSPKILMLTDGSN | |
| | NVSWASMYKVISNVSNNDANALQAAPVNLDDHVTAILCSSGTTGLPKGV | |
| | MLTDKNITTVISIFMNTNTIPENAVSLSLLPFFHAYSFVLMVLGLLKGNCSVV | |
| | FSRFEEKLFLQYIEKYRIEYLPVVPSLMVFLAKHPLVDKYDLSCVKTIWSGA | |
| | APLSKEIQQAVAKRLNITSVKQGYGLTETTLAVLRSPDEKEKLGSVGVIVS | |
| | GMSAKVIPIGEYETDEALGPNCEGELCFKGDLIMKGYYNDEKSTRAIIDKD | |
| | GWLHTGDVGYYDEDGYFFIVDRIKELIKYKGYQVPPAELEAILLTCPGIQDA | |
| | AVIGIPNDKTGELPMAFVVKEENSNICEKDIIQYVNGKNFQYHI | |

A second list of luciferase-like sequences was then generated by using the least identical luciferase sequence from Table 3, Photophorus jansonii (R4WV23) as the query for a new search against UniprotKB database. This ultimately provided luciferase sequences with identities varying from 82% to 38%, as shown in Table 5.

TABLE 5
Selection of insect luciferase from SANSparallel web server
searched against UniprotKB database for sequences homologous
to Photophorus Jansonii (R4WV23) for protein sequence alignment.
Photophorus Jansonii luciferase protein sequence was the most
dissimilar compared to Photinus pyralis in Table 3. A protein sequence
homology search of Photophorus Jansonii was implemented to select
for further luciferase protein sequences for alignment. Luciferases were
chosen whose protein sequence identity differed from that found in
Photophorus Jansonii to approximately 0.4. Care was taken to avoid
repeats of selected species from Table 3.

| | Sequence | |
|---|---|---|
| Organism | Identity | UniProtKB Identifier |
| Photophorus jansonii | 1.0 | R4WV23_9COLE |
| Pyrophorus plagiophthalamus | 0.82 | Q718E3_9COLE |
| Pyrophorus angustus luscus | 0.58 | D2KV87_9COLE |
| Agrypnus binodulus binodulus | 0.54 | A9ZPM4_9COLE |
| Luciola cruciate | 0.52 | E3WI50_LUCCR |
| Phausis reticulata | 0.50 | A0A1B3TNR3_9COLE |
| Anopheles darlingi | 0.45 | W5JL40_ANODA |
| Habropoda laboriosa | 0.45 | A0A0L7R1M1_9HYME |
| Blattella germanica | 0.43 | A0A2P8Y7W7_BLAGE |
| Camponotus floridanus | 0.42 | E2AA43_CAMFO |
| Acromyrmex echinatior | 0.42 | F4WWH1_ACREC |
| Culex quinquefasciatus | 0.41 | B0W9Q1_CULQU |
| Bactrocera latifrons | 0.41 | A0A0K8VAT3_BACLA |
| Trachymyrmex zeteki | 0.41 | A0A151WRK1_9HYME |
| Bombyx mori | 0.38 | G8GE17_BOMMO |
| Papilio machaon | 0.38 | A0A194R162_PAPMA |
| Danaus plexippus plexippus | 0.38 | A0A212FIH9_DANPL |

Referring to Table 6 and FIGS. 7-9, the SANSParallel server provided similar luciferase sequence identities compared to BLASTP for the luciferase sequences that were most distantly related from Photinus pyralis in Tables 3 and 5.

TABLE 6
BLASTP alignment results of first and last luciferase proteins sequences within Table 3 and 5.
For alignment set 1, the first and last luciferase sequences from Table 3 were aligned using
BLASTP. For alignment set 2, the first and last luciferase sequences from Table 5 were aligned
using BLASTP. For alignment set 3, the first luciferase sequence from Table 3 was aligned to
the last luciferase sequence from Table 5 using BLASTP. ‘Identities’ are the percent of amino
acids that are identical, ‘Positives’ are the percent of amino acids either identical or conserved.
‘Gap’ is the percentage of gaps per amino acid generated through alignment of BLASTP.

| Alignment | | | | | |
|---|---|---|---|---|---|
| Set | Organism | UniProtKB Identifier | Identities | Positives | Gaps |
| 1 | Photinus pyralis | H1AD96_PHOPY | 48% | 69% | 0% |
| | (Table 3) | | (259/545) | (380/545) | (5/545) |
| | (SEQ ID NO: 15) | | | | |
| | Photophorus jansonii | R4WV23_9COLE | | | |
| | (Table 3) | | | | |
| | (SEQ ID NO: 19) | | | | |
| 2 | Photophorus jansonii | R4WV23_9COLE | 37% | 54% | 7% |
| | (Table 5) | | (205/555) | (300/555) | (42/555) |
| | (SEQ ID NO: 19) | | | | |
| | Danaus plexippus | A0A212FIH9_DANPL | | | |
| | plexippus (Table 5) | | | | |
| | (SEQ ID NO: 18) | | | | |
| 3 | Photinus pyralis | H1AD96_PHOPY | 33% | 53% | 5% |
| | (Table 3) | | (168/512) | (275/512) | (27/512) |
| | (SEQ ID NO: 15) | | | | |
| | Danaus plexippus | A0A212FIH9_DANPL | | | |
| | plexippus (Table 5) | | | | |
| | (SEQ ID NO: 18) | | | | |

All 35 luciferase sequences from Tables 3 & 5 were aligned using ClustalO, and potential fragmentation sites were chosen for experimental validation on the basis of a lack of homology/dissimilarity that was visually detected amongst the sequences. Sites for fragmentation were chosen where the dissimilarity was also congruent to a solvent accessible loop within the firefly luciferase crystal structure (FIG. 6). From analysis of the sequence dissimilarity and structure, three potential fragmentation sites were identified, Q159, G228, and V374 (FIGS. 1C-1D) that, to the inventors' knowledge, have never been explored for testing the design of split-protein reassembly. Two additional sites, I397 and P405, were also tested.

The three newly identified split-Fluc pairs, as well as the I397 and P405 split-Flucs, were cloned and subsequently tested for complementation by the classic chemically induced dimerization (CID) system using the protein pairs, FK506-binding protein (FKBP) and FKBP rapamycin-binding (FRB) domains. The N-terminal fragment of each split-luciferase was fused to FKBP at the C-terminal end and the C-terminal fragment of each split-luciferase was fused to FRB at the N-terminal end. Referring to FIG. 2A, the linkers between the enzyme fragments and the FKBP or FRB included 27 to 30 residue glycine-serine linkers. With the five split-Fluc constructs in hand, complementation was tested employing a cell-free protein expression approach using rabbit reticulocyte lysate (RRL), which allows for rapid protein expression and analysis. In brief, equal amounts of mRNA of each split-Fluc pair being tested was incubated in RRL with either 100 nM of rapamycin or DMSO for 1.5 h. Following incubation, L-Glo reagent containing the substrate, luciferin, was added to each of the split-Fluc pairs and luminescence was subsequently measured.

As shown in FIG. 2B-2C, all five split-Fluc were shown to provide an increase in luminescence in the presence of added rapamycin, thus verifying the sequence dissimilarity-based approach. Split-Fluc228 displayed the highest overall signal but also showed significant background complementation without added rapamycin. In comparison to split-Fluc228, split-Fluc374 and split-Fluc405 showed higher signal to background ratios, 388 and 803 respectively, but provided lower overall signal, suggesting that either the fragments are potentially misfolded (lower available final active enzyme) or alternatively, catalysis is compromised in the reassembled enzyme. Split-sites 159 and 397 both showed intermediate signal to background, 89 and 206-fold respectively. Thus the sequence dissimilarity approach successfully established three completely new split-sites in Fluc.

Having identified several new split-Flucs, it was another objective to optimize the newly identified split-protein pairs to improve signal over background by structure guided redesign of the interface. To test the design approach for split-protein optimization, split-Fluc228 was chosen as a starting point, which displayed high signal and also high background in the initial experiments. The goal was to inspect the interface and introduce a series of mutations that would reduce unassisted/background complementation while maintaining catalytic proficiency.

Structure-Guided Mutagenesis and Optimization

Referring to Table 7, a detailed inspection of the firefly luciferase structure suggests that the two halves of split-Fluc228 make several potential side chain and backbone interactions ≤3.5 Å. Referring to FIGS. 10A-10B, without wishing to be bound to theory, it was hypothesized that disruption of one or more of these interactions could attenuate the affinity of the split-Fluc228 fragments and reduce background signal without significantly impacting catalytic efficiency.

TABLE 7
List of molecular interactions of split-firefly luciferase residues
subjected to alanine mutagenesis.

| N-Fluc228 | C-Fluc229 | |
|---|---|---|
| Residues | Residues | N-Fluc to C-Fluc Interaction (Type) |
| K8 | E430 | Side Chain-Side Chain (Salt bridge) |
| Y33 | I257 | Side Chain-Backbone (Hydrogen bond) |
| T43 | R261 | Side Chain-Side Chain (Hydrogen bond) |
| H46 | Y266 | Side Chain-Side Chain (π stacking) |
| N50 | R261 | Side Chain-Side Chain (Hydrogen bond) |

Based on the analysis of interactions between the fragments and taking into account the residues hypothesized to be implicated in catalysis, a series of alanine point mutations were made to the N-terminal fragment of split-Fluc228. Each mutation was expected to potentially reduce affinity between fragments but not interfere with catalysis. Interestingly, several of the potential interactions that were targeted localized to a beta-sheet between the fragments of split-Fluc228 as seen in FIG. 3A. All the individual alanine point mutations were introduced in the N-terminal fragment of split-Fluc228 as one of the identified interactions was between the hydroxyl of Y33 on the N-terminal fragment and a backbone carbonyl at position I257 on the C-terminal fragment. Limiting mutations to one fragment limits the number of variables when comparing the split-Fluc228 mutants.

With the split-Fluc228 mutants in hand, the rapamycin induced split-Fluc228 reassembly was tested. Without wishing to be bound to a particular theory, it was hypothesized that the single alanine substitutions would likely reduce affinity between the two fragments, resulting in reduced unassisted complementation. Three mutations in particular, Y33A, H46A. and N50A, significantly reduced unassisted complementation while maintaining strong rapamycin dependent signals. Mutations K8A and T43A reduced unassisted complementation, but also attenuated rapamycin dependent signal and were not chosen for further split-Fluc228 optimization (FIG. 3B).

The first-generation split-Fluc228 redesign suggests that the parallel beta-sheet between the fragments (FIG. 3A) contributes considerably to the observed spontaneous assembly but importantly, changes to these residues did not significantly reduce the observed luminescence activity. These three successful first-generation designs prompted a second-generation design by creating three new double mutant combinations, Y33A/H46A, Y33A/N50A, and H46A/N50A. It was an objective to investigate whether the second-generation mutations would additively reduce split-luciferase background complementation without significantly compromising activity. Thus, three new sets of split-Fluc228 constructs attached to FKBP and FRB were cloned and subsequently tested using cell-free assay. Two of the second-generation split-Fluc228 constructs, split-Fluc228 Y33A/1-146A and split-Fluc228 H46A/N50A, maintained strong CID dependent signal, providing 246-fold and 400-fold signal to background, respectively. Split-Fluc228 Y33A/N50A gave the lowest rapamycin induced signal, however, it still provided a considerable 543-fold signal to background (FIG. 3C). All three designed second-generation split-Fluc228 variants were chosen for further evaluation and characterization in mammalian cells.

New Split-Luciferases in Mammalian Cells

While there are many biophysical approaches to protein-protein interactions in vitro, there are a limited number of direct and robust binding assays in cells. The engineered split-luciferase was evaluated using the same fragment assisted complementation strategy as above (FKBP-Rapamycin-FRB) to determine if it would provide robust signal in mammalian cells.

Equal amounts of the split-luciferase halves were transiently expressed in HEK293T cells, incubated cells with either rapamycin or vehicle for 14 h, and then assayed clarified lysate for luminescence. The relative luminescence levels from equivalent amounts of protein between the three candidates constructs correlated well with the in vitro data. Split-Fluc228 Y33A/H46A displayed the strongest signal, while Y33A/N50A comparatively provided the least overall signal although all constructs provided very good signal over background (FIG. 4B). The fold change in CID dependent luminescence to background complementation was greatest for split-Fluc228 33A/N50A (575×), while split-Fluc228 H46A/N50A split provided strong (376×) but lower signal to background. Split-Fluc228 Y33A/H46A displayed over 5-fold greater absolute rapamycin dependent luminescence in the cell-based assay compared to the other two variants, while still providing excellent signal to background (425×). Thus, the utility of split-Fluc228 Y33A/H46A was further probed in cellular assays.

For a split-protein reporter to be useful, it should provide more than a simple on/off answer and ideally be sensitive to the concentrations of the added small molecule and demonstrate a dose dependent response. Referring to FIG. 5A, the FKBP/FRB appended split-Fluc228 Y33A/H46A was investigated for rapamycin concentration dependent signal. The rapamycin dependent titration clearly demonstrated that inducible luminescence is dose dependent and saturable in HEK293T cells. The titration of rapamycin provides an apparent dissociation constant (K_{d}) of 10 nM. Protein concentrations of the fragments are not known in these types of assays and only relative ligand affinities can be estimated under very similar experimental conditions.

Finally, with the successful second-generation split-Fluc228 Y33A/H46A in hand, its use in monitoring interacting proteins other than FKBP/FRB was investigated. To this end, Bcl-2 protein family interactions and their response to small molecule inhibitors were evaluated. The Bcl-2 family of proteins is critical in regulating a cell's commitment to apoptosis and essential in orchestrating a number of cellular processes such as development and stress response. Moreover, deregulation of Bcl-2 family interactions have been implicated in a variety of cancers and this class of PPIs has been widely targeted with what are now classic small molecule PPI inhibitors. The Bcl-2-like protein 1 (Bcl-xL) and the BH3-domain of Bcl-2 antagonist killer 1 (BAK) were selected as the interacting protein pair due to their well characterized interaction and the availability of small molecule inhibitors. ABT-737, which is known to bind to Bcl-xL and inhibit the Bcl-xL/BAK interaction, was chosen as the small molecule inhibitor.

Split-Fluc228 Y33A/H46A constructs were created by appending the soluble domain of Bcl-xL to the C-terminal fragment of N-Fluc228 Y33A/H46A with a 27-residue glycine-serine linker, giving N-Fluc228 Y33A/H46A-GS27-Bcl-xL. The BH3 domain of BAK (20 residues) was appended to the N-terminal fragment of C-Fluc229 through a 30-residue glycine-serine linker, providing BAK-GS30-C-Fluc229. Equal amounts of the Bcl-xL and BAK appended split-Fluc fragments were transiently expressed in HEK293T cells, and subsequently incubated cells with varying concentration of ABT-737 or vehicle for 14 h, then assayed the clarified lysate for luminescence. Referring to Table 8, split-Fluc228 Y33A/H46A with Bcl-xL-BAK interacting pairs displayed excellent signal to background (486×) in cells treated with DMSO compared to cells treated with 10 μM ABT-737. More importantly, titration with ABT-737 was clearly dose dependent and provided an IC₅₀ value of 4 nM as shown in FIG. 5B, which is in the relevant affinity range for ABT-737.

TABLE 8
In cellulo (HEK293T cells) ABT-737 mediated inhibition luminescence values of N-Fluc228
Y33A/H46A-Bcl-xL CΔ24 with BAK-CFIuc229. Cells were incubated with ABT-737 for 14 hours.

| | DMSO | | 10 μM ABT-737 | | | |
|---|---|---|---|---|---|---|
| N-Fluc228 | Average | Standard | Average | Standard | Percent | Signal over |
| Mutations | RLU | Deviation | RLU | Deviation | Inhibition | Background |
| 228 WT | 11,086,202 | 854,454 | 438,145 | 88,992 | 96.1% | 25 |
| Y33/H46A | 1,097,797 | 59,015 | 2,258 | 110 | 99.8% | 486 |

RLU = Relative Light Units

It is important that the split-Fluc228 Y33A/H46A system recapitulates full inhibition (>99%) of Bcl-xL interaction with BAK by ABT-737 in mammalian cells. These results help confirm that the new split-Fluc228 Y33A/H46A variant can be used to monitor and effectively evaluate PPIs and test for their inhibitors in mammalian cells. Thus, these final sets of experiments with the new split-Fluc demonstrate an ability to effectively monitor biologically relevant PPIs in mammalian cells and also an ability to interrogate inhibitors of PPIs in their native environment.

As used herein, the term “about” refers to plus or minus 10% of the referenced number.

Although there has been shown and described a preferred embodiment of the present invention, it will be readily apparent to those skilled in the art that modifications may be made thereto which do not exceed the scope of the appended claims. Thus, the scope of the invention is only to be limited by the following claims. In some embodiments, the figures in this patent application are drawn to scale, including the angles, ratios of dimensions, etc. Alternatively, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of preparing a split signal-generating protein, comprising:
a) identifying a fragmentation site in a signal-generating protein using sequence dissimilarity analysis;
b) splitting the signal-generating protein at the fragmentation site to produce a first protein fragment and a second protein fragment; and
c) mutating at least one residue in one or both of the protein fragments.

2. The method of claim 1, further comprising:
a) connecting the first protein fragment to a first protein of a protein-protein interaction via a first linker to form a first sensor complex; and
b) connecting the second protein fragment to a second protein of the protein-protein interaction via a second linker to form a second sensor complex, wherein the split-protein sensor comprises the first and second sensor complexes.

3. The method of claim 2, wherein the method generates a split-protein sensor for detecting protein-protein interactions.

4. The method of claim 1, wherein the signal-generating protein is a luminescent protein.

5. The method of claim 1, wherein mutating at least one residue comprises replacing said residue with another amino acid.

6. The method of claim 1, wherein the signal-generating protein has a signal to background ratio that is greater than 200.

7. A method of detecting protein-protein interactions, comprising:
a) preparing a split-protein sensor comprising:
i) analyzing a signal-generating protein using sequence dissimilarity to identify a fragmentation site in said protein;
ii) splitting the signal-generating protein at the fragmentation site to produce a first protein fragment and a second protein fragment;
iii) mutating at least one residue in one or both of the protein fragments;
iv) connecting the first protein fragment to a first protein of a protein-protein interaction via a first linker to form a first sensor complex; and
v) connecting the second protein fragment to a second protein of the protein-protein interaction via a second linker to form a second sensor complex, wherein the split-protein sensor comprises the first and second sensor complexes; and
b) detecting a signal from the split-protein sensor when the first protein and the second protein interact, wherein the protein-protein interaction causes the first protein fragment and the second protein fragment to associate, thereby reassembling the signal-generating protein that exhibits a detectable signal.

8. The method of claim 7, wherein the signal-generating protein is a luminescent protein.

9. The method of claim 8, wherein the luminescent protein exhibits a detectable luminescence signal.

10. The method of claim 7, wherein the protein-protein interaction is caused by the first and second proteins binding to a target molecule.

11. The method of claim 10, wherein the target molecule is a biological molecule, drug compound, or other chemical compound.

12. A split-protein sensor for detecting protein-protein interactions, comprising:
a) a first sensor complex comprising a first protein of the protein-protein interaction linked to a first protein fragment; and
b) a second sensor complex comprising a second protein of the protein-protein interaction linked to a second protein fragment;
wherein the first and second protein fragments are derived from splitting of a fragmentation site of a signal-generating protein, wherein the fragmentation site is determined by sequence dissimilarity, wherein one or both of the protein fragments have at least one mutated residue, wherein interaction of the first protein and the second protein causes reassembly of the signal-generating protein which exhibits a detectable signal.

13. The sensor of claim 12, wherein the first protein fragment and the second protein fragment are derived from splitting a fragmentation site between residues GN of a sequence DPIFGNQIIPDT (SEQ ID NO: 6) in firefly Photinus pyralis luciferase (H1AD96).

14. The sensor of claim 12, wherein the first protein fragment has a mutation at the Y residue of a loop in Photinus pyralis luciferase comprising the sequence RYALVPGT (SEQ ID NO: 9) and at an H residue of another loop comprising the sequence AHIEV (SEQ ID NO: 10), wherein said Y and H residues are replaced with A.

15. The sensor of claim 13, wherein interaction of the first protein and second protein causes re-assembly of the firefly Photinus pyralis luciferase, which exhibits a detectable luminescence.

16. The sensor of claim 12, wherein the signal-generating protein is a luminescent protein.

17. The sensor of claim 16, wherein the luminescent protein exhibits a detectable luminescence signal.

18. The sensor of claim 12, wherein the split-protein sensor has a signal to background ratio that is greater than 200.

19. The sensor of claim 12, wherein the protein-protein interaction is caused by the first protein and second protein binding to a target molecule.

20. The sensor of claim 19, wherein the target molecule is a biological molecule, drug compound, or other chemical compound.
